SEQUENCE LISTING (37 sequences)

SEQ ID NO: 1 - protein, 5 aa, Artificial Sequence
  Description of Artificial Sequence Synthetic peptide
RRXXX

SEQ ID NO: 2 - protein, 7 aa, Artificial Sequence
  Description of Artificial Sequence Synthetic peptide
LRRASLG

SEQ ID NO: 3 - protein, 6 aa, Artificial Sequence
  Description of Artificial Sequence Synthetic peptide
RXXXFF

SEQ ID NO: 4 - protein, 7 aa, Artificial Sequence
  Description of Artificial Sequence Synthetic peptide
RQGSFRA

SEQ ID NO: 5 - protein, 4 aa, Artificial Sequence
  Description of Artificial Sequence Synthetic peptide
XPXX

SEQ ID NO: 6 - protein, 4 aa, Artificial Sequence
  Description of Artificial Sequence Synthetic peptide
PXXP

SEQ ID NO: 7 - protein, 7 aa, Artificial Sequence
  Description of Artificial Sequence Synthetic peptide
RRIPLSP

SEQ ID NO: 8 - protein, 9 aa, Artificial Sequence
  Description of Artificial Sequence Synthetic peptide
KKKKRFSFK

SEQ ID NO: 9 - protein, 8 aa, Artificial Sequence
  Description of Artificial Sequence Synthetic peptide
XRXXSXRX

SEQ ID NO: 10 - protein, 9 aa, Artificial Sequence
  Description of Artificial Sequence Synthetic peptide
LRRLSDSNF

SEQ ID NO: 11 - protein, 10 aa, Artificial Sequence
  Description of Artificial Sequence Synthetic peptide
KKLNRTLTVA

SEQ ID NO: 12 - protein, 7 aa, Artificial Sequence
  Description of Artificial Sequence Synthetic peptide
EEIYXXF

SEQ ID NO: 13 - protein, 8 aa, Artificial Sequence
  Description of Artificial Sequence Synthetic peptide
EEIYGEFR

SEQ ID NO: 14 - protein, 6 aa, Artificial Sequence
  Description of Artificial Sequence Synthetic peptide
EIYEXX

SEQ ID NO: 15 - protein, 6 aa, Artificial Sequence
  Description of Artificial Sequence Synthetic peptide
IYMFFF

SEQ ID NO: 16 - protein, 4 aa, Artificial Sequence
  Description of Artificial Sequence Synthetic peptide
YMMM

SEQ ID NO: 17 - protein, 5 aa, Artificial Sequence
  Description of Artificial Sequence Synthetic peptide
EEEYF

SEQ ID NO: 18 - protein, 12 aa, Artificial Sequence
  Description of Artificial Sequence Synthetic peptide
RIGEGTYGVVRR

SEQ ID NO: 19 - protein, 7 aa, Artificial Sequence
  Description of Artificial Sequence Synthetic peptide
RPRTSSF

SEQ ID NO: 20 - protein, 7 aa, Artificial Sequence
  Description of Artificial Sequence Synthetic peptide
PRTPGGR

SEQ ID NO: 21 - protein, 8 aa, Artificial Sequence
  Description of Artificial Sequence Synthetic peptide
RLNRTLSV

SEQ ID NO: 22 - protein, 8 aa, Artificial Sequence
  Description of Artificial Sequence Synthetic peptide
DRRLSSLR

SEQ ID NO: 23 - protein, 12 aa, Artificial Sequence
  Description of Artificial Sequence Synthetic peptide
EAIYAAPFARRR

SEQ ID NO: 24 - protein, 15 aa, Artificial Sequence
  Description of Artificial Sequence Synthetic peptide
KVEKIGEGTYGVVYK

SEQ ID NO: 25 - protein, 8 aa, Artificial Sequence
  Description of Artificial Sequence Synthetic peptide
EEEIYGEF

SEQ ID NO: 26 - protein, 12 aa, Artificial Sequence
  Description of Artificial Sequence Synthetic peptide
RHSSPHQSEDEE

SEQ ID NO: 27 - protein, 18 aa, Artificial Sequence
  Description of Artificial Sequence Synthetic peptide
RRKDLHDDEEDEAMSITA

SEQ ID NO: 28 - protein, 4 aa, Artificial Sequence
  Description of Artificial Sequence Synthetic peptide
SXXX

SEQ ID NO: 29 - protein, 4 aa, Artificial Sequence
  Description of Artificial Sequence Synthetic peptide
SXXX

SEQ ID NO: 30 - protein, 10 aa, Artificial Sequence
  Description of Artificial Sequence Synthetic peptide
RRRDDDSDDD

SEQ ID NO: 31 - protein, 17 aa, Artificial Sequence
  Description of Artificial Sequence Synthetic peptide
KGPWLEEEEEAYGWLDF

SEQ ID NO: 32 - protein, 7 aa, Artificial Sequence
  Description of Artificial Sequence Synthetic peptide
EEIYGEF

SEQ ID NO: 33 - protein, 8 aa, Artificial Sequence
  Description of Artificial Sequence Synthetic peptide
RREIYGRF

SEQ ID NO: 34 - protein, 7 aa, Artificial Sequence
  Description of Artificial Sequence Synthetic peptide
RRIYGRF

SEQ ID NO: 35 - protein, 7 aa, Artificial Sequence
  Description of Artificial Sequence Synthetic peptide
PXSPXSP

SEQ ID NO: 36 - protein, 6 aa, Artificial Sequence
  Description of Artificial Sequence Synthetic peptide
PFHLVI

SEQ ID NO: 37 - protein, 8 aa, Artificial Sequence
  Description of Artificial Sequence Synthetic peptide
KEEIYGEF

(19) United States
(12) Patent Application Publication
(10) Pub. No.: US 2006/0121553 A1
(43) Pub. Date: Jun. 8, 2006

(54) Fluorogenic enzyme assay methods, kits and compositions using charge-balancers

(75) Inventor: Hongye Sun, San Mateo, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/261,173

(22) Filed: Oct. 27, 2005

Related U.S. Application Data
(60) Provisional application No. 60/623,363, filed on Oct. 29, 2004.

(51) Int. Cl. C12Q 1/37; C07K 7/08; C07K 7/06

(52) U.S. Cl. 435/23; 530/329; 530/330

(57) ABSTRACT

Fluorescent compositions, methods and kits useful for, among other things, detecting, quantifying and/or characterizing enzymes.

### 1. CROSS REFERENCES TO RELATED APPLICATIONS

This application claims benefit under 35 U.S.C. § 119(e) to application Ser. No. 60/623,363, entitled “Fluorogenic Enzyme Assay Methods, Kits and Compositions Using Charge-Balancers,” filed Oct. 29, 2004; the disclosure of which is incorporated herein by reference in its entirety.

### 2. FIELD

The present disclosure relates to compositions, methods and kits for detecting, quantifying and/or characterizing enzymes in a sample.

### 3. INTRODUCTION

Enzymes are molecules that increase the rate of chemical reactions. Enzymatic assays for detecting, quantifying and/or characterizing enzyme activity have significant biological, medical and industrial applications. In biological systems, enzymes are involved in synthesis and replication of nucleic acids, modification, and degradation of polypeptides, synthesis of metabolites, and many other functions. In medical testing, enzymes are important indicators of the health or disease of human patients. In industry, enzymes are used for many purposes, such as proteases used in laundry detergents, metabolic enzymes to make specialty chemicals such as amino acids and vitamins, and chirally specific enzymes to prepare enantiomerically pure drugs. Assays using reporter molecules are important tools for studying and detecting enzymes that mediate numerous biological and industrial processes. Although numerous approaches have been developed for assaying enzymes using reporter molecules, there remains a great need to find new assay designs that can be used to inexpensively and conveniently detect and characterize a wide variety of enzymes.

### 4. SUMMARY

Provided herein are compositions, methods and kits useful for, among other things, detecting, quantifying and/or characterizing enzymes. The compositions generally comprise one or more molecules that collectivity include four different types of moieties: a hydrophobic moiety, a fluorescent moiety, a substrate moiety and a charge-balance moiety when included in an aqueous solvent at or above its critical micelle concentration (CMC). The fluorescent moiety functions to produce a fluorescent signal when the substrate moiety of the composition is acted upon by an enzyme. The substrate moiety comprises a substrate or putative substrate for an enzyme of interest. The charge-balance moiety acts to balance the overall charge of the composition. While not intending to be bound by any theory of operation, it is believed that balancing the overall net charge acts to promote or encourage micelle formation.

The hydrophobic, fluorescent, substrate, and charge-balance moieties can be included in a single molecule, or they can be included in different molecules. As a specific example, in some embodiments, the composition comprises a substrate molecule that comprises a hydrophobic moiety capable of integrating the substrate molecule into a micelle, a fluorescent moiety, a substrate moiety, and a charge-balance moiety. As another specific example, in some embodiments, the composition comprises two distinct molecules, a substrate molecule and a charge-balance molecule. In some embodiments, the substrate molecule comprises a hydrophobic moiety and a substrate moiety. The charge-balance molecule comprises a hydrophobic moiety and a charge-balance moiety. The hydrophobic moieties are selected such that they, either individually or together, are capable of integrating the substrate molecule and the charge-balance molecule into a micelle. The hydrophobic moieties comprising the various molecules can be the same, some of them can be the same and others different, or they may all differ from another. For example, in some embodiments the hydrophobic moieties comprising the substrate molecule and the charge-balance molecule can be the same. In other embodiments, the hydrophobic moieties comprising the substrate molecule and the charge-balance molecule can differ from each other.

One or both of the substrate and/or charge-balance molecules further comprises a fluorescent moiety. Non-limiting examples of suitable fluorescent dyes that can comprise the fluorescent moiety(ies) include xanthene dyes such as fluorescein, sulfofluorescein and rhodamine dyes, cyanine dyes, bodipy dyes and squaraine dyes. Fluorescent moieties comprising other fluorescent dyes may also be used.

The various substrate and/or charge-balance molecules can comprise additional moieties. As a specific example, a substrate molecule can comprise a charge-balance moiety and vice-versa. As another specific example, the compositions can comprise a quenching moiety. The quenching moiety can be included in the substrate molecule, the charge-balance molecule, in both the substrate molecule and charge-balance molecule, or in a distinct quenching molecule. In some embodiments, a quenching molecule comprises a hydrophobic moiety and a quenching moiety. The quenching moiety can be any moiety capable of quenching the fluorescence of a fluorescent moiety when the quenching moiety is in close proximity to the fluorescent moiety.

These and other features of the present teachings are set forth below.

### 5. BRIEF DESCRIPTION OF THE DRAWINGS

The skilled artisan will understand that the drawings, described below, are for illustration purposes only. The drawings are not intended to limit the scope of the present teaching in any way.

FIG. 1 illustrates an exemplary embodiment of an enzyme assay scheme utilizing an exemplary embodiment of a single molecule comprising a hydrophobic moiety, a fluorescent moiety, a substrate moiety and a charge-balance moiety.

FIG. 2 illustrates an exemplary embodiment of an enzyme assay scheme utilizing an exemplary embodiment of a substrate molecule and a charge-balance molecule.

FIG. 3 illustrates an exemplary embodiment of an enzyme assay scheme utilizing an exemplary embodiment of a substrate molecule, charge-balance molecule and a quenching molecule.

FIGS. 4A-D illustrate exemplary embodiments of substrate molecules comprising a hydrophobic moiety, a charge-balance moiety(ies), a fluorescent moiety, and a substrate moiety.

FIGS. 5A-H illustrate exemplary embodiments of substrate molecules (FIGS. 5A, C, E, G) and charge-balance molecules (FIGS. 5B, D, F, H).

FIGS. 6A-B illustrate exemplary embodiments of a substrate molecule (FIG. 6A) and a charge-balance molecule (FIG. 6B).

FIG. 7 shows the addition of varying concentrations (0, 5, 10, 20, 50 μM) of a charge-balance molecule, C₁₆RROOORRIYGRF quenching the fluorescence of a substrate molecule, C₁₆K(Dye2)OOOEEIYGEF (10 μM) in 25 mM Tris (pH 7.6).

FIG. 8 shows the rate of reaction of 5 nM tyrosine kinase (Lyn) against the substrate molecule C₁₆K(Dye2)OOOEEIYGEF (2 μM), charge-balance molecule C₁₆RROOORRIYGRF (2 μM), with 0 and 100 μM ATP.

### 6. DESCRIPTION OF VARIOUS EMBODIMENTS

It is to be understood that both the foregoing summary and the following description of various embodiments are exemplary and explanatory only and are not restrictive of the present teachings. In this application, the use of the singular includes the plural unless specifically stated otherwise. Also, the use of “or” means “and/or” unless stated otherwise. Similarly, “comprise,” “comprises,” “comprising,” “include,” “includes” and “including” are not intended to be limiting.

6.1 Definitions

As used herein, the following terms and phrases are intended to have the following meanings:

“Detect” and “detection” have their standard meaning, and are intended to encompass detection, measurement, and characterization of a selected enzyme or enzyme activity. For example, enzyme activity can be “detected” in the course of detecting, screening for, or characterizing inhibitors, activators, and modulators of the enzyme activity.

“Fatty Acid” has its standard meaning and is intended to refer to a long-chain hydrocarbon carboxylic acid in which the hydrocarbon chain is saturated, mono-unsaturated or polyunsaturated. The hydrocarbon chain can be linear, branched or cyclic, or can comprise a combination of these features, and can be unsubstituted or substituted. Fatty acids typically have the structural formula RC(O)OH, where R is a substituted or unsubstituted, saturated, mono-unsaturated or polyunsaturated hydrocarbon comprising from 6 to 30 carbon atoms which has a structure that is linear, branched, cyclic or a combination thereof.

“Micelle” has its standard meaning and is intended to refer to an aggregate formed by amphipathic molecules in water or an aqueous environment such that their polar ends or portions are in contact with the water or aqueous environment and their nonpolar ends or portions are in the interior of the aggregate. A micelle can take any shape or form, including but not limited to, a non-lamellar “detergent-like” aggregate that does not enclose a portion of the water or aqueous environment, or a unilamellar or multilamellar “vesicle-like” aggregate that encloses a portion of the water or aqueous environment, such as, for example, a liposome.

“Quench” has its standard meaning and is intended to refer to a reduction in the fluorescence intensity of a fluorescent group or moiety as measured at a specified wavelength, regardless of the mechanism by which the reduction is achieved. As specific examples, the quenching can be due to molecular collision, energy transfer such as FRET, photoinduced electron transfer such as PET, a change in the fluorescence spectrum (color) of the fluorescent group or moiety or any other mechanism (or combination of mechanisms). The amount of the reduction is not critical and can vary over a broad range. The only requirement is that the reduction be detectable by the detection system being used. Thus, a fluorescence signal is “quenched” if its intensity at a specified wavelength is reduced by any measurable amount. A fluorescence signal is “substantially quenched” if its intensity at a specified wavelength is reduced by at least 50%, for example by 50%, 60%, 70%, 75%, 80%, 85%, 90%, 95%, 96%, 97%, 98%, 99% or even 100%.

Polypeptide sequences are provided with an orientation (left to right) of the N terminus to C terminus, with amino acid residues represented by the standard 3-letter or 1-letter codes (e.g., Stryer, L., Biochemistry, 2^{nd} Ed., W.H. Freeman and Co., San Francisco, Calif., page 16 (1981)).

6.2 Compositions

Provided herein are compositions, methods and kits useful for, among other things, detecting, quantifying and/or characterizing enzymes. The compositions typically form micelles comprising one or more molecules that collectively include a number of different moieties, such as a hydrophobic moiety, a fluorescent moiety, a substrate moiety, and a charge-balance moiety. The hydrophobic moieties are capable of anchoring or integrating the molecules into the micelle. The exact numbers, lengths, sizes and/or composition of the hydrophobic moieties can be varied. In embodiments employing two distinct molecules, each hydrophobic moiety may be the same, or some or all of the hydrophobic moieties may differ.

The substrate moiety comprises substrate that can be acted upon by a specific enzyme or agent. The fluorescence signal of the fluorescent moiety is quenched when the substrate molecule and/or the charge-balance molecule is integrated into the micelle. When the substrate moiety is acted upon by the specified enzyme it promotes the dissociation of the fluorescent moiety from the micelle, thereby reducing or eliminating the quenching effect caused by the interactions between the fluorescent moiety and the micelle. The dissociation may be caused by cleavage of the enzyme recognition site or by the addition, deletion, or substitution of chemical groups, such as phosphate groups, which can destabilize the substrate molecule in the micelle, promoting its release therefrom. Release of the fluorescent moiety from the micelle reduces or eliminates the quenching effect, thereby producing a detectable increase in fluorescence.

The charge-balance moiety acts to balance the overall charge of the micelle. For example, if the substrate molecule comprises one or more charged chemical groups, the presence of these groups can destabilize the substrate molecule in the micelle, thereby promoting the release of the substrate molecule from the micelle in the absence of the specified enzyme. Release of the charged substrate molecule from the micelle can be prevented by including a charge-balance moiety designed to counter the charge of the substrate molecule via the inclusion of chemical groups that have the opposite charge of the chemical groups comprising the substrate molecule, such that the overall charge of the micelle is neutral. Thus, by including the charge-balance moiety, stable micelles can be formed in the presence of destabilizing chemical groups. When the substrate moiety is acted upon by the specified enzyme it promotes destabilization of the micelle, for example, by the addition of charged groups, and dissociation of the fluorescent moiety from the micelle, thereby reducing or eliminating the quenching effect and producing a detectable increase in fluorescence.

In some embodiments, the micelle comprises a single molecule that includes a hydrophobic moiety, a fluorescent moiety, a substrate moiety and a charge-balance moiety. In other embodiments, the micelle comprises two distinct molecules, a substrate molecule and a charge-balance molecule. For example, in some embodiments, the substrate molecule comprises a hydrophobic moiety and a substrate moiety. The charge-balance molecule comprises a hydrophobic moiety and a charge-balance moiety. One or both of the substrate molecule and/or charge-balance molecule further comprises a fluorescent moiety. The moieties can be connected to each other in any way that permits them to perform their respective functions.

In other embodiments, the micelle can comprise additional molecules such as a quenching molecule. The quenching molecule can include a hydrophobic moiety and a quenching moiety that quenches the fluorescence of the fluorescent moiety. The quenching moiety can be positioned so that it is able to intramolecularly quench the fluorescence of the fluorescent moiety on the substrate molecule and/or the charge-balance molecule, which includes it, or, alternatively, the quenching moiety may be positioned so that intramolecular quenching does not occur. In either embodiment, the quenching moiety may intermolecularly quench the fluorescence of a fluorescent moiety on another molecule in the micelle which is in close proximity thereto. When the substrate moiety of the substrate molecule is acted upon by a specified enzyme it “deactivates” the quenching effect by relieving the close proximity of the quenching and fluorescent moieties, thereby generating a measurable increase in fluorescence signals.

6.3 The Hydrophobic Moiety

The hydrophobic moiety(ies) act to anchor or integrate the various molecules described herein into the micelle. The exact numbers, lengths, size and/or compositions of the hydrophobic moieties can be varied. For example, in embodiments employing two or more hydrophobic moieties, each hydrophobic moiety may be the same, or some or all of the hydrophobic moieties may differ. As a specific example, in some embodiments, the composition comprises two distinct molecules, a substrate molecule and a charge-balance molecule, each which can comprise a hydrophobic moiety. In some embodiments, the hydrophobic moiety(ies) of the substrate molecule may be the same length, size and/or composition from the hydrophobic moiety(ies) of the charge-balance molecule. In some embodiments, the hydrophobic moiety(ies) of the substrate molecule may differ in length, size and/or composition from the hydrophobic moiety(ies) of the charge-balance molecule.

In some embodiments, the hydrophobic moieties comprise a substituted or unsubstituted hydrocarbon of sufficient hydrophobic character (e.g., length and/or size) to cause the substrate molecule and/or the charge-balance molecule to become integrated or incorporated into a micelle when the molecule(s) is placed in an aqueous environment at a concentration above a micelle-forming threshold, such as at or above its critical micelle concentration (CMC). In other embodiments, the hydrophobic moieties comprise a substituted or unsubstituted hydrocarbon comprising from 6 to 30 carbon atoms, or from 6 to 25 carbon atoms, or from 6 to 20 carbon atoms, or from 6 to 15 carbon atoms, or from 8 to 30 carbon atoms, or from 8 to 25 carbon atoms, or from 8 to 20 carbon atoms, or from 8 to 15 carbon atoms, or from 12 to 30 carbon atoms, or from 12 to 25 carbon atoms, or from 12 to 20 carbon atoms. The hydrocarbon can be linear, branched, cyclic, or any combination thereof, and can optionally include one or more of the same or different substituents. Exemplary linear hydrocarbon groups comprise C6, C7, C8, C9, C10, C11, C12, C13, C14, C15, C16, C17, C18, C19, C20, C22, C24, and C26 alkyl chains.

In some embodiments, the hydrophobic moieties are fully saturated. In some embodiments, the hydrophobic moieties can comprise one or more carbon-carbon double bonds which can be, independently of one another, in the cis or trans configuration, and/or one or more carbon-carbon triple bonds. In some cases, the hydrophobic moieties can have one or more cycloalkyl groups, or one or more aryl rings or arylalkyl groups, such as one or two phenyl rings.

In some embodiments, the hydrophobic moiety is a nonaromatic moiety that does not have a cyclic aromatic pi electron system. In some embodiments, if the hydrophobic moiety contains one or more unsaturated carbon-carbon bonds, those carbon-carbon bonds are not conjugated. In another embodiment, the structure of the hydrophobic moiety is incapable of interacting with the fluorescent moiety, by a FRET or stacking interaction, to quench fluorescence of the fluorescent moiety. Also encompassed herein are embodiments that involve a combination of any two or more of the foregoing embodiments. Optimization testing can be done by making several substrate and/or charge-balance molecules having different hydrophobic moieties.

In some embodiments, the molecule(s) of the composition comprises two hydrophobic moieties linked to the C1 and C2 carbons of a glycerolyl group via ester linkages (or other linkages). The two hydrophobic moieties can be the same or they can differ from another. In a specific embodiment, each hydrophobic moiety is selected to correspond to the hydrocarbon chain or “tail” of a naturally occurring fatty acid. In another specific embodiment, the hydrophobic moieties are selected to correspond to the hydrocarbon chains or tails of a naturally occurring phospholipid. Non-limiting examples of hydrocarbon chains or tails of commonly occurring fatty acids are provided in Table 1, below:

TABLE 1

| Length:Number of | |
|---|---|
| Unsaturations | Common Name |
| 14:0 | myristic acid |
| 16:0 | palmitic acid |
| 18:0 | stearic acid |
| 18:1 cisΔ⁹ | oleic acid |
| 18:2 cisΔ^{9,12} | linoleic acid |
| 18:3 cisΔ^{9,12,15} | linonenic acid |
| 20:4 cisΔ^{5,8,11,14} | arachidonic acid |
| 20:5 cisΔ^{5,8,11,14,17} | eicosapentaenoic acid (an omega-3 fatty acid) |

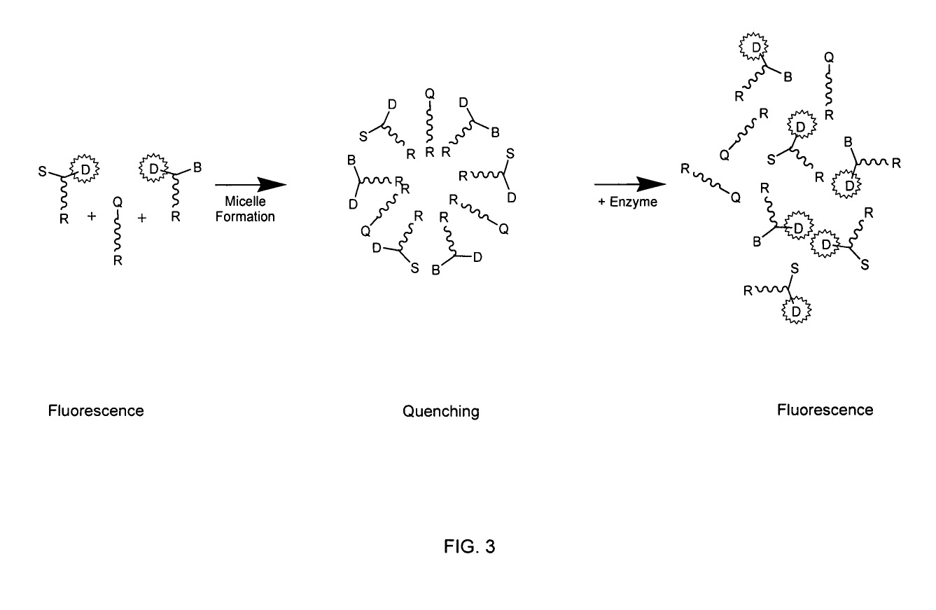

In some embodiments, the hydrophobic moieties comprise amino acids or amino acid analogs that have hydrophobic side chains. The amino acids or analogs are chosen to provide sufficient hydrophobicity to integrate the molecule(s) of the composition into a micelle under the assay conditions used to detect the enzymes. Exemplary hydrophobic amino acids include alanine, glycine, valine, leucine, isoleucine, proline, phenylalanine, methionine, tryptophan, and cysteine as described in Alberts, B., et al., Molecular Biology of the Cell, 4^{th} Ed., Garland Science, New York, N.Y., FIG. 3.3 (2002)). Exemplary amino acid analogs include norvaline, aminobutyric acid, cyclohexylalanine, butylglycine, phenylglycine, and N-methylvaline (see “Amino Acids and Amino Acid Analogs” section 2002-2003 Novabiochem catalog).

The hydrophobicity of a hydrophobic moiety can be calculated by assigning each amino acid a hydrophobicity value and then averaging the values along the hydrophobic moiety. Hydrophobicity values for the common amino acids are shown Table 2.

TABLE 2
Hydrophobicity of Amino Acids

| | Monera et al.¹ | Hopp-Woods² | Kyte-Doolittle³ |
|---|---|---|---|
| Amino Acid | Hydrophobicity at | Hydrophobicity | Hydrophobicity |
| (IUPAC) | pH 7 | scale | scale |
| Alanine (A) | 41 | −0.5 | −1.8 |
| Cysteine (C) | 49 | −1.0 | −2.5 |
| Aspartic acid (D) | −55 | 3.0 | 3.5 |
| Glutamic acid (E) | −31 | 3.0 | 3.5 |
| Phenylalanine (F) | 100 | −2.5 | −2.8 |
| Glycine (G) | 0 | 0.0 | 0.4 |
| Histidine (H) | 8 | −0.5 | 3.2 |
| Isoleucine (I) | 99 | −1.8 | −4.5 |
| Lysine (K) | −23 | 3.0 | 3.9 |
| Leucine (L) | 97 | −1.8 | −3.8 |
| Methionine (M) | 74 | −1.3 | −1.9 |
| Asparagine (N) | −28 | 0.2 | 3.5 |
| Proline (P) | −46 (pH 2) | 0.0 | 1.6 |
| Glutamine (Q) | −10 | 0.2 | 3.5 |
| Arginine (R) | −14 | 3.0 | 4.5 |
| Serine (S) | −5 | 0.3 | 0.8 |
| Threonine (T) | 13 | −0.4 | 0.7 |
| Valine (V) | 76 | −1.5 | −4.2 |
| Tryptophan (W) | 97 | −3.4 | 0.9 |
| Tyrosine (Y) | 63 | −2.3 | 1.3 |

¹Monera et al. J. Protein Sci 1: 219-329 (1995) (The values were normalized so that the most hydrophobic residue (phenylalanine) is given a value of 100 relative to glycine, which is considered neutral (0 value)).
²Hoop TP and Woods KR: Prediction of protein antigenic determinants from amino acid sequences. Proc Natl Acad Sci USA 78: 3824, 1981.
³Kyte J and Doolittle RF: A simple method for displaying the hydropathic character of a protein. J Mol Biol 157: 105, 1982.

The exact number of amino acids or amino acid analogs chosen will vary depending on the sequence of the amino acids selected and the presence of other constituents. In some embodiments, the hydrophobic moiety comprises the same amino acid or amino acid analog. For example, the hydrophobic moiety can comprise poly(leucine) from 1 and 10 leucine residues. In some embodiments, the hydrophobic moiety comprises a mixture of amino acids or amino acid analogs. For example, the hydrophobic moiety can comprise a mixture of amino acids, such as leucine and isoleucine, from 1 to 10 leucine resides and from 1 to 10 isoleucine residues can be used.

In some embodiments, the hydrophobic moiety can comprise a mixture of amino acids, amino acid analogs, and hydrocarbons. For example, in some embodiments, the hydrophobic moiety can comprise from 1 to 10 residues of the amino acids or amino acid analogs and a hydrocarbon comprising from 2 to 30 carbons atoms.

The hydrophobic moieties can be connected to the other moieties comprising the substrate molecule and/or the charge-balance molecule in any way that permits them to perform their respective functions. For example, if the substrate molecule comprises the hydrophobic moiety, the fluorescent moiety, the substrate moiety and the charge-balance moiety, the moieties can be connected directly to one another, i.e., covalently linked to each other. In other embodiments, one, some, or all of the moieties can be connected indirectly to one another, i.e., via one or more optional linkers.

For embodiments of molecule(s) of the compositions in which the hydrophobic moiety is linked to the fluorescent moiety (discussed below), it will be understood that the hydrophobic moiety is distinct from the fluorescent moiety because the hydrophobic moiety does not comprise any of the atoms in the fluorescent moiety that are part of the aromatic or conjugated pi-electron system that produces the fluorescent signal. Thus, if a hydrophobic moiety is connected to the C4 position of a xanthene ring (e.g., the C4′ position of a fluorescein or rhodamine dye), the hydrophobic moiety does not comprise any of the aromatic ring atoms of the xanthene ring.

6.4 The Fluorescent Moiety

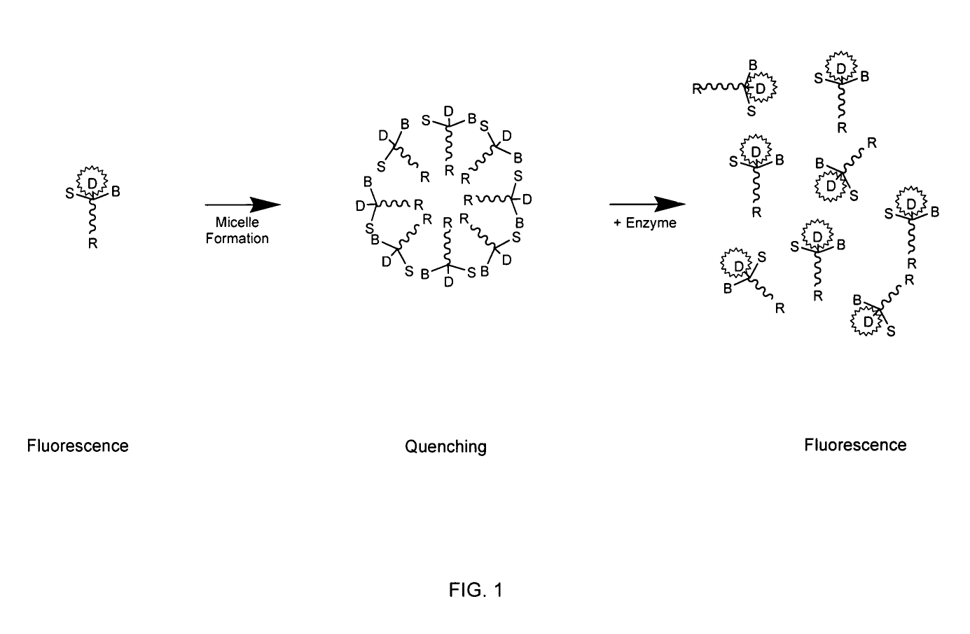

The substrate molecule and/or the charge-balance molecule can further comprise one or more fluorescent moiety(ies) which can be selectively “turned on” when the substrate molecule and/or micelle is acted upon by an enzyme or agent as described herein. The fluorescent moiety can comprise any entity that provides a fluorescent signal and that can be used in accordance with the methods and principles described herein. In the exemplary embodiment illustrated in FIG. 1, the fluorescence of the fluorescent moiety is quenched when the substrate molecule is incorporated into the micelle. When the substrate moiety is acted upon by a specified enzyme it results in the dissociation of the substrate molecule and/or micelle resulting in the release of the fluorescent moiety, thereby increasing the fluorescent signal produced by the fluorescent moiety.

The fluorescent moiety(ies) can be connected to the other moieties comprising the substrate molecule and/or the charge-balance molecule in any way that permits them to perform their respective functions. For example, if the substrate molecule comprises the hydrophobic moiety, the fluorescent moiety, the substrate moiety and the charge-balance moiety, the moieties can be connected directly to one another, i.e., covalently linked to each other. In other embodiments, one, some or all of the moieties can be connected indirectly to one another, i.e., via one or more optional linkers.

Quenching of the fluorescent moiety within the micelle can be achieved in a variety of different ways. In one embodiment, the quenching effect may be achieved or caused by “self-quenching.” Self-quenching can occur when the substrate molecule and/or the charge-balance molecule comprising a fluorescent moiety are present in the micelle at a concentration sufficient or molar ratio high enough to bring their fluorescent moieties in close enough proximity to one another such that their fluorescence signals are quenched. Release of the fluorescent moieties from the micelle reduces or abolishes the “self-quenching,” producing an increase in their fluorescence signals. As used herein, a fluorescent moiety is “released” or “removed” from a micelle if any molecule or molecular fragment that contains the fluorescent moiety is released or removed from the micelle.

For any given assay, the fluorescent moiety can be soluble or insoluble. For example, in some embodiments the fluorescent moiety is soluble under conditions of the assay so as to facilitate removal of the released fluorescent moiety from the micelle into the assay medium. In other embodiments, provided that self-quenching does not occur, the fluorescent moiety is insoluble under conditions of the assay so that the fluorescent moiety can precipitate out of solution and localize at the site at which it was produced, thereby producing an increase in the fluorescent signal as compared to the signal observed in solution.

The quenching effect can be achieved or caused by other moieties comprising the micelle. These moieties are referred to as “quenching moieties,” regardless of the mechanism by which the quenching is achieved. Such quenching moieties and quenching molecules are described in more detail, below. By modifying the quenching moieties to reduce or eliminate their quenching effects, or by removing the fluorescent moiety from proximity of the quenching moieties, the fluorescence of the fluorescent moiety can be substantially restored. Any mechanism that is capable of causing quenching or changes in fluorescence properties may be used in the micelles and methods described herein.

The degree of quenching achieved within the micelle is not critical for success, provided that it is measurable by the detection system being used. As will be appreciated, higher degrees of quenching are desirable, because the greater the quenching effect, the lower the background fluorescence prior to removal of the quenching effect. In theory, a quenching effect of 100%, which corresponds to complete suppression of a measurable fluorescence signal, would be ideal. In practice, any measurable amount will suffice. The amount and/or molar percentage of substrate molecule and/or the charge-balance molecule and optional quenching molecule in a micelle necessary to provide a desired degree of quenching in the micelle may vary depending upon, among other factors, the choice of the fluorescent moiety. The amount and/or molar percentage of any substrate molecule and/or the—balance molecule (or mixture of substrate molecules and/or the charge-balance molecules) and optional quenching molecule (or mixture of optional quenching molecules) comprising a substrate molecule and/or the charge-balance molecule-containing micelle in order to obtain a sufficient degree of quenching can be determined empirically.

Typically, the fluorescent moiety of the substrate molecule and/or the charge-balance molecule comprises a fluorescent dye that in turn comprises a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, such as xanthenes, rhodamines, fluoresceins, cyanines, phthalocyanines, squaraines, bodipy dyes, coumarins, oxazines, and carbopyronines.

In some embodiments, the fluorescent moiety comprises a xanthene dye. Generally, xanthene dyes are characterized by three main features: (1) a parent xanthene ring; (2) an exocyclic hydroxyl or amine substituent; and (3) an exocyclic oxo or imminium substituent. The exocyclic substituents are typically positioned at the C3 and C6 carbons of the parent xanthene ring, although “extended” xanthenes in which the parent xanthene ring comprises a benzo group fused to either or both of the C5/C6 and C3/C4 carbons are also known. In these extended xanthenes, the characteristic exocyclic substituents are positioned at the corresponding positions of the extended xanthene ring. Thus, as used herein, a “xanthene dye” generally comprises one of the following parent rings:

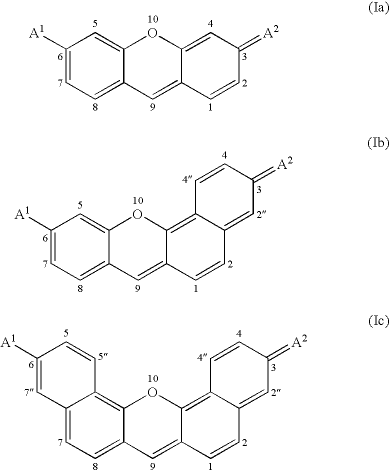

In the parent rings depicted above, A¹ is OH or NH₂ and A² is O or NH₂⁺. When A¹ is OH and A² is O, the parent ring is a fluorescein-type xanthene ring. When A¹ is NH₂ and A² is NH₂⁺, the parent ring is a rhodamine-type xanthene ring. When A¹ is NH₂ and A² is O, the parent ring is a rhodol-type xanthene ring.

One or both of nitrogens of A¹ and A² (when present) and/or one or more of the carbon atoms at positions C1, C2, C2″, C4, C4″, C5, C5″, C7″, C7 and C8 can be independently substituted with a wide variety of the same or different substituents. In one embodiment, typical substituents comprise, but are not limited to, —X, —R^{a}, —OR^{a}, —SR^{a}, —NR^{a}R^{a}, perhalo (C₁-C₆) alkyl, —CX₃, —CF₃, —CN, —OCN, —SCN, —NCO, —NCS, —NO, —NO₂, —N₃, —S(O)₂O, —S(O)₂OH, —S(O)₂R^{a}, —C(O)R, —C(O)X, —C(S)R^{a}, —C(S)X, —C(O)OR^{a}, —C(O)O⁻, —C(S)OR^{a}, —C(O)SR^{a}, —C(S)SR^{a}, —C(O)NR^{a}R^{a}, —C(S)NR^{a}R^{a} and —C(NR)NR^{a}R^{a}, where each X is independently a halogen (preferably —F or —Cl) and each R^{a} is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, 5-20 membered heteroaryl, 6-26 membered heteroarylalkyl, 5-20 membered heteroaryl-heteroaryl, carboxyl, acetyl, sulfonyl, sulfinyl, sulfone, phosphate, or phosphonate. Generally, substituents which do not tend to completely quench the fluorescence of the parent ring are preferred, but in some embodiments quenching substituents may be desirable. Substituents that tend to quench fluorescence of parent xanthene rings are electron-withdrawing groups, such as —NO₂, —Br and —I.

The C1 and C2 substituents and/or the C7 and C8 substituents can be taken together to form substituted or unsubstituted buta[1,3]dieno or (C₅-C₂₀) aryleno bridges. For purposes of illustration, exemplary parent xanthene rings including unsubstituted benzo bridges fused to the C1/C2 and C7/C8 carbons are illustrated below:

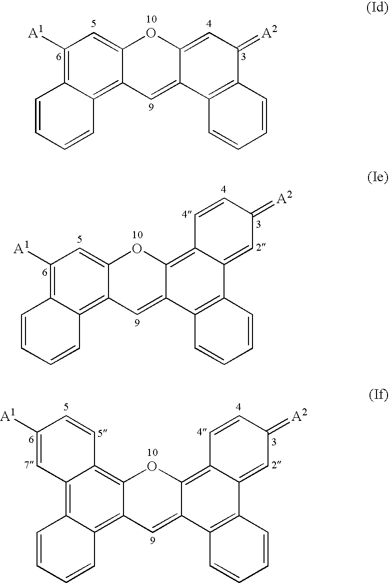

The benzo or aryleno bridges may be substituted at one or more positions with a variety of different substituent groups, such as the substituent groups previously described above for carbons C1-C8 in structures (Ia)-(Ic), supra. In embodiments including a plurality of substituents, the substituents may all be the same, or some or all of the substituents can differ from one another.

When A¹ is NH₂ and/or A² is NH₂⁺, the nitrogen atoms may be included in one or two bridges involving adjacent carbon atom(s). The bridging groups may be the same or different, and are typically selected from (C₁-C₁₂) alkyldiyl, (C₁-C₁₂) alkyleno, 2-12 membered heteroalkyldiyl and/or 2-12 membered heteroalkyleno bridges. Non-limiting exemplary parent rings that comprise bridges involving the exocyclic nitrogens are illustrated below:

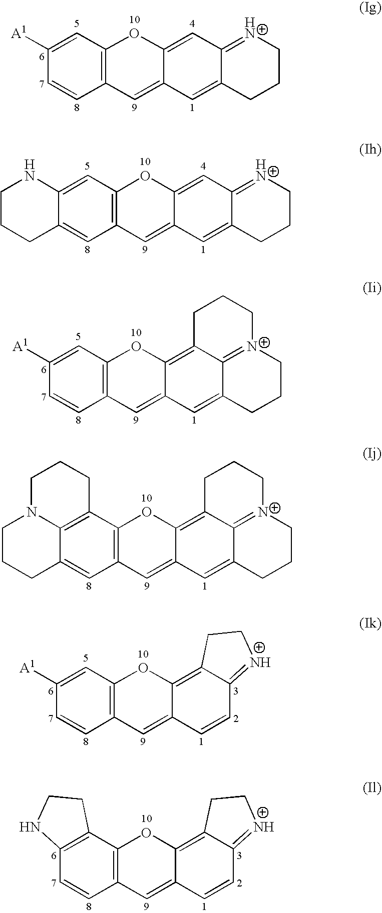

The parent ring may also comprise a substituent at the C9 position. In some embodiments, the C9 substituent is selected from acetylene, lower (e.g., from 1 to 6 carbon atoms) alkanyl, lower alkenyl, cyano, aryl, phenyl, heteroaryl, electron-rich heteroaryl and substituted forms of any of the preceding groups. In embodiments in which the parent ring comprises benzo or aryleno bridges fused to the C1/C2 and C7/C8 positions, such as, for example, rings (Id), (le) and (If) illustrated above, the C9 carbon is preferably unsubstituted.

In some embodiments, the C9 substituent is a substituted or unsubstituted phenyl ring such that the xanthene dye comprises one of the following structures:

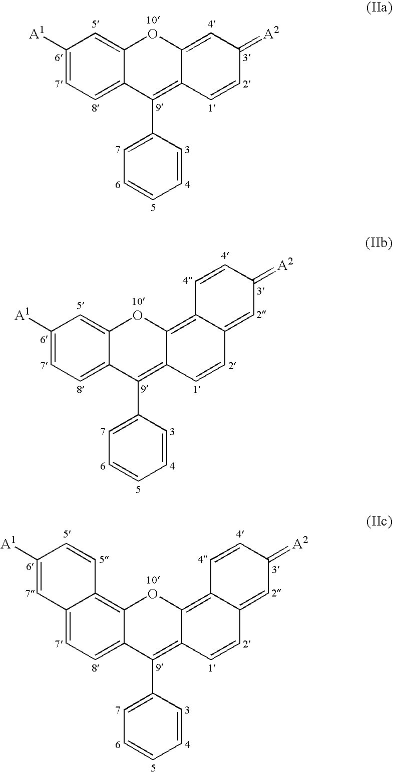

The carbons at positions 3, 4, 5, 6 and 7 may be substituted with a variety of different substituent groups, such as the substituent groups previously described for carbons C1-C8. In some embodiments, the carbon at position C3 is substituted with a carboxyl (—COOH) or sulfuric acid (—SO₃H) group, or an anion thereof. Dyes of formulae (Ia), (IIb) and (IIc) in which A¹ is OH and A² is 0 are referred to herein as fluorescein dyes; dyes of formulae (IIa), (IIb) and (IIc) in which A¹ is NH₂ and A² is NH₂⁺ are referred to herein as rhodamine dyes; and dyes of formulae (IIa), (IIb) and (IIc) in which A¹ is OH and A² is NH₂⁺ (or in which A¹ is NH₂ and A² is O) are referred to herein as rhodol dyes.

As highlighted by the above structures, when xanthene rings (or extended xanthene rings) are included in fluorescein, rhodamine and rhodol dyes, their carbon atoms are numbered differently. Specifically, their carbon atom numberings include primes. Although the above numbering systems for fluorescein, rhodamine and rhodol dyes are provided for convenience, it is to be understood that other numbering systems may be employed, and that they are not intended to be limiting. It is also to be understood that while one isomeric form of the dyes are illustrated, they may exist in other isomeric forms, including, by way of example and not limitation, other tautomeric forms or geometric forms. As a specific example, carboxy rhodamine and fluorescein dyes may exist in a lactone form.

In some embodiments, the fluorescent moiety comprises a rhodamine dye. Exemplary suitable rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional suitable rhodamine dyes include, for example, those described in U.S. Pat. Nos. 6,248,884, 6,111,116, 6,080,852, 6,051,719, 6,025,505, 6,017,712, 5,936,087, 5,847,162, 5,840,999, 5,750,409, 5,366,860, 5,231,191, and 5,227,487; PCT Publications WO 97/36960 and WO 99/27020; Lee et al., NUCL. ACIDS RES. 20:2471-2483 (1992), Arden-Jacob, NEUE LANWELLIGE XANTHEN-FARBSTOFFE FÜR FLUORESZENZSONDEN UND FARBSTOFF LASER, Verlag Shaker, Germany (1993), Sauer et al., J. FLUORESCENCE 5:247-261 (1995), Lee et al., NUCL. ACIDS RES. 25:2816-2822 (1997), and Rosenblum et al., NUCL. ACIDS RES. 25:4500-4504 (1997). A particularly preferred subset of rhodamine dyes are 4,7,-dichlororhodamines. In one embodiment, the fluorescent moiety comprises a 4,7-dichloro-orthocarboxyrhodamine dye.

In some embodiments, the fluorescent moiety comprises a fluorescein dye. Exemplary suitable fluorescein include, but are not limited to, fluorescein dyes described in U.S. Pat. Nos. 6,008,379, 5,840,999, 5,750,409, 5,654,442, 5,188,934, 5,066,580, 4,933,471, 4,481,136 and 4,439,356; PCT Publication WO 99/16832, and EPO Publication 050684. A preferred subset of fluorescein dyes are 4,7-dichlorofluoresceins. Other preferred fluorescein dyes include, but are not limited to, 5-carboxyfluorescein (5-FAM) and 6-carboxyfluorescein (6-FAM). In one embodiment, the fluorescein moiety comprises a 4,7-dichloro-orthocarboxyfluorescein dye.

In some embodiments, the fluorescent moiety can include a cyanine, a phthalocyanine, a squaraine, or a bodipy dye, such as those described in the following references and the references cited therein: U.S. Pat. Nos. 6,080,868, 6,005,113, 5,945,526, 5,863,753, 5,863,727, 5,800,996, and 5,436,134; and PCT Publication WO 96/04405.

In some embodiments, the fluorescent moiety can comprise a network of dyes that operate cooperatively with one another such as, for example by FRET or another mechanism, to provide large Stoke's shifts. Such dye networks typically comprise a fluorescence donor moiety and a fluorescence acceptor moiety, and may comprise additional moieties that act as both fluorescence acceptors and donors. The fluorescence donor and acceptor moieties can comprise any of the previously described dyes, provided that dyes are selected that can act cooperatively with one another. In a specific embodiment, the fluorescent moiety comprises a fluorescence donor moiety which comprises a fluorescein dye and a fluorescence acceptor moiety which comprises a fluorescein or rhodamine dye. Non-limiting examples of suitable dye pairs or networks are described in U.S. Pat. Nos. 6,399,392, 6,232,075, 5,863,727, and 5,800,996.

6.5 The Substrate Moiety

The substrate molecule comprises one or more substrate moieties that can be acted upon by enzymes or agents. In some embodiments, the substrate molecule comprises one substrate moiety. In some embodiments, the substrate molecule comprises two, three, four, or more substrate moieties, wherein the substrate moieties can be the same or different. The substrate moieties can be connected in any way that permits them to perform their respective function. In some embodiments, the substrate moieties can be directly connected to each other. In other embodiments, the substrate moieties can be indirectly connected to each other via one or more linkage groups. In yet other embodiments, the substrate moieties are indirectly linked to each other through the fluorescent moiety or the hydrophobic moiety.

In some embodiments, the “substrate moiety” or “protein recognition moiety” or “recognition moiety” includes all or a subset of the residues comprising the substrate or the consensus sequence for a specified enzyme. For example, for a protein kinase, the total number of residues comprising the substrate moiety is defined by N, wherein N is an integer from 1 to 10. In some embodiments, N is an integer from 1 to 15. In other embodiments, N is an integer from 1 to 20. As a specific example of these embodiments, the consensus sequence for PKA is—R—R—X—S/T-Z, thus, N=5. Repetition of the consensus sequence, two, three, or four, or more times can be used to provide a kinase substrate with two, three, four or more unphosphorylated residues.

In other embodiments, the substrate moiety comprises a subset of residues comprising the substrate or consensus sequence for a specified enzyme. In these embodiments, one or more residues are omitted from the substrate or consensus sequence. A subset is defined herein as comprising N—u amino acid residues, wherein, as defined above, N represents the total number of amino acid residues comprising the substrate or consensus sequence, and u represents the number of amino acid residues omitted from the substrate or consensus sequence. In some embodiments, u is an integer from 1 to 9. In other embodiments, u is an integer from 1 to 14. In still other embodiments, u is an integer from 1 to 19. For example, if the total number of amino acids in the substrate is 4, subsets comprising 3, 2, or 1 amino acid residue(s) can be made. If the total number of amino acids in the substrate is 5, subsets comprising 4, 3, 2, or 1 amino acid residue(s) can be made. If the total number of amino acids in the substrate is 6, subsets comprising 5, 3, 2, or 1 amino acid residue(s) can be made. If the total number of amino acids in the substrate is 7, subsets comprising 6, 5, 4, 3, 2, or 1 amino acids residue(s) can be made. If the substrate comprises 8 amino acids, subsets comprising 7, 6, 5, 4, 3, 2, or 1 amino acid residue(s) can be made. If the total number of amino acids in the substrate is 9, subsets comprising 8, 7, 6, 5, 4, 3, 2, or 1 amino acids residue(s) can be made. If the substrate comprises 10 amino acids, subsets comprising 9, 8, 7, 6, 5, 4, 3, 2, or 1 amino acids residue(s) can be made. Typically, subsets comprising N−1 or N-2 amino acid residues are made.

In other embodiments, one substrate moiety can share one or more residues comprising the substrate or consensus sequence. As a specific example of these embodiments, the substrate for p38βII is P—X—S—P. One substrate moiety with an overlapping residue can be created, wherein one residue of the first substrate P—X—S—P is shared with the second substrate X—S—P, such that one substrate moiety, comprising overlapping substrates i.e., P—X—S—P—X—S—P is formed. Non-limiting examples of suitable substrate moieties comprising two, three four or more consensus sequences are described in U.S. application Ser. No. 11/158,525, filed Jun. 21, 2005, and entitled “Kinase Substrates with Multiple Phosphorylation Sites.”

In some embodiments, two or more substrate moieties can share one or more residues comprising the substrate or consensus sequence. In these embodiments, residues from one substrate moiety are included in another substrate moiety.

A substrate moiety comprises a substrate or putative substrate that can be acted upon by specified enzymes or agents. Any type of enzyme or chemical reactions on the substrate moiety/micelle may be used, provided that it is capable of producing a detectable change (e.g., an increase) in fluorescence. Preferably, the specified enzyme is substantially active at the interface between the micelle and the assay medium. Selection of a particular enzyme or chemical reaction on the substrate moiety, and hence substrate moiety, may depend, in part, on the structure of the substrate molecule, as well as on other factors.

In some embodiments, the enzyme or agent act upon the substrate moiety to cleave the substrate moiety. In these embodiments, the substrate moiety comprises a cleavage site that is cleavable by a chemical reagent or cleaving enzyme. As a specific example, the substrate moiety can comprise a cleavage site that is cleavable by a lipase, a phospholipase, a peptidase, a nuclease or a glycosidase enzyme. The substrate moiety may further comprise additional residues and/or features that facilitate the specificity, affinity and/or kinetics of the cleaving enzyme. Depending upon the requirements of the particular cleaving enzyme, such cleaving enzyme “recognition moieties” can comprise the cleavage site or, alternatively, the cleavage site may be external to the recognition moiety. For example, certain endonucleases cleave at positions that are upstream or downstream of the region of the nucleic acid molecule bound by the endonuclease.

The chemical composition of the substrate moiety will depend upon, among other factors, the requirements of the cleaving enzyme. For example, if the cleaving enzyme is a protease, the substrate moiety can comprise a peptide (or analog thereof) recognized and cleaved by the particular protease. If the cleaving enzyme is a nuclease, the substrate moiety can comprise an oligonucleotide (or analog thereof) recognized and cleaved by a particular nuclease. If the cleaving enzyme is a phospholipase, the substrate moiety can comprise a diacylglycerolphosphate group recognized and cleaved by a particular phospholipase.

Sequences and structures recognized and cleaved by the various different types of cleaving enzymes are well known. Any of these sequences and structures can comprise the substrate moiety. Although the cleavage can be sequence specific, in some embodiments it can be non-specific. For example, the cleavage can be achieved through the use of a non-sequence specific nuclease, such as, for example, an RNase.

Cleavage of the substrate moiety of the substrate molecule by the corresponding cleaving enzyme can release the fluorescent moiety from the micelle, reducing or eliminating its quenching and producing a measurable increase in fluorescence.

In other embodiments, the enzyme or agent acts upon the substrate moiety by the addition, deletion, or substitution of chemical moieties to the substrate moiety. These reactions can destabilize the substrate molecule in the micelle, thereby promoting its release from the micelle. The release of the substrate molecule increases the fluorescence of its fluorescent moiety.

As a specific example, in some embodiments, the enzyme or agent acts upon the substrate moiety to change the net charge of the substrate moiety, such as by phosphorylation of one or more unphosphorylated residues by a kinase enzyme or dephosphorylation of one or more phosphorylated residues by a phosphatase enzyme. Specific examples of substrate molecules comprising substrate moieties modifiable by protein kinase and phosphatase enzymes are described in more detail, below.

By way of illustration, the substrate moiety is first discussed below with reference to protein kinases as exemplary enzymes to be detected, quantified, and/or characterized. In addition to playing important biochemical roles, protein kinases are also useful for illustrating enzymes that cause an increase in the net charge of an substrate moiety by adding a phosphate group to a hydroxyl group to form a phosphorylated substrate moiety. Under physiological conditions, i.e. pH 6 to pH 8, phosphorylation of the substrate moiety causes the addition of two negative charges, for a net change in charge of −2. Enzymes that carry out the opposite reaction, protein phosphatases, are also discussed, which cause a net increase in charge of ⁺2 in the substrate moiety, under physiological conditions, i.e. pH 6 to pH 8. In either case, the amplitude of the net charge on the substrate moiety is increased. For example, upon phosphorylation of a substrate moiety as described above, the amplitude of the net negative charge on the substrate molecule is increased by ⁻2. On the other hand, upon dephosphorylation of a substrate moiety by a phosphatase, the amplitude of the net positive charge on the substrate molecule is increased by ⁺2.

In some embodiments, a protein kinase substrate moiety for detecting, quantifying and/or characterizing one or more protein kinases in a sample is provided. In some compositions, a substrate molecule comprises a hydrophobic moiety capable of integrating the substrate molecule into a micelle, a substrate moiety comprising a protein kinase substrate moiety comprising a unphosphorylated residue that is capable of being phosphorylated by a protein kinase, a fluorescent moiety and a charge-balance moiety such that the net charge of the micelle ranges from ⁻1 to ⁺1 at physiological pH.

In another exemplary class of compositions, a micelle comprises (i) a substrate molecule that comprises a hydrophobic moiety capable of integrating the substrate molecule into the micelle, a substrate moiety comprising a protein kinase substrate moiety comprising a unphosphorylated residue that is capable of being phosphorylated by a protein kinase and an optional fluorescent moiety; and (ii) a charge-balance molecule that comprises a hydrophobic moiety capable of integrating the charge-balance molecule into the micelle, a charge-balance moiety capable of balancing the overall charge of the micelle, such that the net charge of the micelle ranges from ⁻1 to ⁺1 at physiological pH. The optional fluorescent moiety can be part of the substrate molecule, charge-balance molecule, or both.

The protein kinase substrate moiety generally comprises an amino acid side chain containing a group that is capable of being phosphorylated by a protein kinase. In some embodiments, the phosphorylatable group is a hydroxyl group. Usually, the hydroxyl group is provided as part of a side chain in a tyrosine, serine, or threonine residue, although any other natural or non-natural amino acid side chain or other entity containing a phosphorylatable hydroxyl group can be used. The phosphorylatable group can also be a nitrogen atom, such as the nitrogen atom in the epsilon amino group of lysine, an imidazole nitrogen atom of histidine, or a guanidinium nitrogen atom of arginine. The phosphorylatable group can also be a carboxyl group in an asparate or glutamate residue.

The protein kinase substrate moiety can further comprise a segment, typically a polypeptide segment, that contains one or more subunits or residues (in addition to the phosphorylatable residue) that impart identifying features to the substrate to make it compatible with the substrate specificity of the protein kinase(s) to be detected, quantified, and/or characterized.

A variety of protein kinase recognitions moieties suitable for use in substrate molecules described herein are taught in copending application No. 60/582,038, filed Jun. 21, 2004, the disclosure of which is incorporated herein by reference.

A wide variety of protein kinases have been characterized over the past several decades, and numerous classes have been identified (see, e.g., S. K. Hanks et al., Science 241:42-52 (1988); B. E. Kemp and R. B. Pearson, Trends Biochem. Sci. 15:342-346 (1990); S. S. Taylor et al., Ann. Rev. Cell Biol. 8:429-462 (1992); Z. Songyang et al., Current Biology 4:973-982 (1994); and Chem. Rev. 101:2209-2600, “Protein Phosphorylation and Signaling” (2001)). Exemplary classes of protein kinases include cAMP-dependent protein kinases (also called the protein kinase A family, A-proteins, or PKA's), cGMP-dependent protein kinases, protein kinase C enzymes (PKC's, including calcium dependent PKC's activated by diacylglycerol), Ca²⁺/calmodulin-dependent protein kinase I or II, protein tyrosine kinases (e.g., PDGF receptor, EGF receptor, and Src), mitogen activated protein (MAP) kinases (e.g., ERK1, KSS1, and MAP kinase type I), cyclin-dependent kinases (CDk's, e.g., Cdk2 and Cdc2), and receptor serine kinases (e.g., TGF-β). Exemplary consensus sequences and/or enzyme substrates for various protein kinases are shown in Table 3, below. As will be appreciated by a person skilled in the art, these various consensus sequences and enzyme substrates can be used to design protein kinase recognition moieties having desired specificities for particular kinases and/or kinase families.

TABLE 3

| | | Consensus Sequence^{a}/ |
|---|---|---|
| Symbol | Description | Enzyme Substrates |
| PKA | cAMP-dependent | -R-R-X-S/T-Z- |
| | | (SEQ ID NO:1) |
| | | -L-R-R-A-S-L-G- |
| | | (SEQ ID NO:2) |
| PhK | phosphorylase | -R-X-X-S/T-F-F- |
| | kinase | (SEQ ID NO:3) |
| | | -R-Q-G-S-F-R-A- |
| | | (SEQ ID NO:4) |
| cdk2 | cyclin-dependent | -S/T-P-X-R/K |
| | kinase-2 | (SEQ ID NO:5) |
| ERK2 | extracellular- | -P-X-S/T-P |
| | regulated kinase-2 | (SEQ ID NO:6) |
| | | -R-R-I-P-L-S-P |
| | | (SEQ ID NO:7) |
| PKC | protein kinase C | K-K-K-K-R-F-S-F-K^{b} |
| | | (SEQ ID NO:8) |
| | | X-R-X-X-S-X-R-X |
| | | (SEQ ID NO:9) |
| CaMKI | Ca²⁺/calmodulin- | L-R-R-L-S-D-S-N-F^{c} |
| | dependent protein | (SEQ ID NO:10) |
| | kinase I | |
| CaMKII | Ca²⁺/calmodulin- | K-K-L-N-R-T-L-T-V-A^{d} |
| | dependent protein | (SEQ ID NO:11) |
| | kinase II | |
| c-Src | cellular form of | -E-E-I-Y-E/G-X-F |
| | Rous sarcoma virus | (SEQ ID NO:12) |
| | transforming agent | -E-E-I-Y-G-E-F-R |
| | | (SEQ ID NO:13) |
| v-Fps | transforming agent | -E-I-Y-E-X-I/V |
| | of Fujinami sarcoma | (SEQ ID NO:14) |
| | virus | |
| Csk | C-terminal Src | -I-Y-M-F-F-F |
| | kinase | (SEQ ID NO:15) |
| InRK | Insulin receptor | -Y-M-M-M |
| | kinase | (SEQ ID NO:16) |
| EGFR | EGF receptor | -E-E-E-Y-F |
| | | (SEQ ID NO:17) |
| SRC | Src kinase | -R-I-G-E-G-T-Y-G-V-V-R-R- |
| | | (SEQ ID NO:18) |
| Akt | RAC-beta serine/ | -R-P-R-T-S-S-F- |
| | threonine- | (SEQ ID NO:19) |
| | protein kinase | |
| Erk1 | Extracellular | -P-R-T-P-G-G-R- |
| | signal-regulated | (SEQ ID NO:20) |
| | kinase 1 (MAP | |
| | kinase 1, MAPK 1) | |
| MAPKAP | MAP kinase- | -R-L-N-R-T-L-S-V |
| K2 | activated protein | (SEQ ID NO:21) |
| | kinase 2 | |
| NEK2 | Serine/threonine- | -D-R-R-L-S-S-L-R |
| | protein | (SEQ ID NO:22) |
| | kinase Nek2 | |
| Ab1 | tyrosine kinase | -E-A-I-Y-A-A-P-F-A-R-R-R |
| | | (SEQ ID NO:23) |
| YES | Proto-oncogene | E-E-I-Y-G-E-F-R |
| | tyrosine-protein | (SEQ ID NO:13) |
| | kinase YES | |
| LCK | Proto-oncogene | E-E-I-Y-G-E-F-R |
| | tyrosine-protein | (SEQ ID NO:13) |
| | kinase LCK | |
| SRC | Proto-oncogene | K-V-E-K-I-G-E-G-T-Y-G-V-V-Y-K |
| | tyrosine-protein | (SEQ ID NO:24) |
| | kinase Src | |
| LYN | Tyrosine-protein | E-E-E-I-Y-G-E-F |
| | kinase LYN | (SEQ ID NO:25) |
| BTK | Tyrosine-protein | E-E-I-Y-G-E-F-R- |
| | kinase BTK | (SEQ ID NO:13) |
| GSK3 | Glycogen synthase | R-H-S-S-P-H-Q-(Sp)-E-D-E-E |
| | kinase-3 | (SEQ ID NO:26) |
| CKI | Casein kinase I | R-R-K-D-L-H-D-D-E-E-D-E-A-M-S-I-T-A |
| | | (SEQ ID NO:27) |
| CKII | Casein kinase II | -(Sp)-X-X-S/T- (SEQ ID NO:28) |
| | | S-X-X-E/D (SEQ ID NO:29) |
| | | R-R-R-D-D-D-S-D-D-D (SEQ ID NO:30) |
| TK | Tyrosine kinase | K-G-P-W-L-E-E-E-E-E-A-Y-G-W-L-D-F |
| | | (SEQ ID NO:31) |

^{a}see, for example, B.E. Kemp and R.B. Pearson, Trends Biochem. Sci. 15:342-346 (1990); Z. Songyang et al., Current Biology 4:973-982 (1994); J.A. Adams, Chem Rev. 101:2272 (2001) and references cited therein; X means any amino acid residue, “/”indicates alternate residues; and Z is a hydrophobic amino acid, such as valine, leucine or isoleucine
^{b}Graff et al., J. Biol. Chem. 266:14390-14398 (1991)
^{c}Lee et al., Proc. Natl. Acad. Sci. 91:6413-6417 (1994)
^{d}Stokoe et al., Biochem. J. 296:843-849 (1993).

Protein kinase substrate moieties having desired specificities for particular kinases and/or kinase families can also be designed, for example, using the methods and/or exemplary sequences described in Brinkworth et al., Proc. Natl. Acad. Sci. USA 100(1):74-79 (2003).

Typically, the protein kinase substrate moieties comprise a sequence of L-amino acid residues. However, any of a variety of amino acids with different backbone or sidechain structures can also be used, such as: D-amino acid polypeptides, alkyl backbone moieties joined by thioethers or sulfonyl groups, hydroxy acid esters (equivalent to replacing amide linkages with ester linkages), replacing the alpha carbon with nitrogen to form an aza analog, alkyl backbone moieties joined by carbamate groups, polyethyleneimines (PEIs), and amino aldehydes, which result in polymers composed of secondary amines. A more detailed backbone list includes N-substituted amide (—CON(R)— replaces —CONH— linkages), esters (—CO₂—), keto-methylene (—COCH₂—) methyleneamino (—CH₂NH—), thioamide (—CSNH—), phosphinate (—PO₂RCH₂—), phosphonamidate and phosphonamidate ester (—PO₂RNH₂), retropeptide (—NHC(O)—), trans-alkene (—CR═CH—), fluoroalkene (e.g.; —CF═CH—), dimethylene (—CH₂CH₂—), thioether (e.g.; —CH₂SCH₂—), hydroxyethylene (—CH(OH)CH₂—), methyleneoxy (—CH₂O—), tetrazole (—CN₄—), retrothioamide (—NHC(S)—), retroreduced (—NHCH₂—), sulfonamido (—SO₂NH—), methylenesulfonamido (—CHRSO₂NH—), retrosulfonamide (—NHS(O₂)—), and peptoids (N-substituted glycines), and backbones with malonate and/or gem-diaminoalkyl subunits, for example, as reviewed by M. D. Fletcher et al., Chem. Rev. 98:763 (1998) and the references cited therein. Peptoid backbones (N-substituted glycines) can also be used (e.g., H. Kessler, Angew. Chem. Int. Ed. Engl. 32:543 (1993); R. N. Zuckermann, Chemtracts-Macromol. Chem. 4:80 (1993); and Simon et al., Proc. Natl. Acad. Sci. 89:9367 (1992)).

In another aspect, a phosphatase substrate moiety for detecting, quantifying, and/or characterizing one or more protein phosphates in a sample is provided. In some compositions, a substrate molecule comprises a hydrophobic moiety capable of integrating the substrate molecule into a micelle, a substrate moiety comprising a phosphatase substrate moiety comprising a phosphorylated residue that is capable of being dephosphorylated by a phosphatase, a fluorescent moiety and a charge-balance moiety capable of balancing the overall charge of the micelle, such that the net charge of the micelle ranges from −1 to +1 at physiological pH.

In another exemplary class of compositions, a micelle comprises (i) a substrate molecule that comprises a hydrophobic moiety capable of integrating the substrate molecule into the micelle, a substrate moiety comprising a phosphatase substrate moiety comprising phosphorylated residue that is capable of being dephosphorylated by a phosphatase and an optional fluorescent moiety; and (ii) a charge-balance molecule that comprises a hydrophobic moiety capable of integrating the charge-balance molecule into the micelle, a charge-balance moiety capable of balancing the overall charge of the micelle, such that the net charge of the micelle ranges from −1 to +1 at physiological pH. The optional fluorescent moiety can be part of the substrate molecule, the charge-balance molecule, or both.

The phosphatase to be detected or characterized can be any phosphatase known in the art. In some embodiments, the phosphate can be a phosphatase 2C, an alkaline phosphatase, or a tyrosine phosphatase. Also, the phosphatase can be a phosphatase candidate, and the methods used to confirm and/or characterize the phosphatase activity of the candidate.

A wide variety of protein phosphatases have been identified (e.g., see P. Cohen, Ann. Rev. Biochem. 58:453-508 (1989), Molecular Biology of the Cell, 3rd edition, Alberts et al., eds., Garland Publishing, NY (1994), and Chem. Rev. 101:2209-2600, “Protein Phosphorylation and Signaling” (2001)). Serine/threonine protein phosphatases represent a large class of enzymes that reverse the action of protein kinase A enzymes, for example. The serine/threonine protein phosphatases have been divided among four groups designated I, IIA, IIB, and IIC. Protein tyrosine kinases are also an important class of phosphatases, and histidine, lysine, arginine, and aspartate phosphatases are also known (e.g., see P. J. Kennelly, Chem Rev. 101:2304-2305 (2001) and references cited therein). In some cases, phosphatases are highly specific for only one or a few proteins, but in other cases, phosphatases are relatively non-specific and can act on a large range of protein targets. Accordingly, the phosphatase substrates of the present teachings can be designed to detect particular phosphatases by suitable selection of the phosphatase recognition moiety. Examples of peptide sequences that can be dephosphorylated by phosphatase activity are described in P. J. Kennelly, Chem. Rev. 101:2291-2312 (2001). Any of the exemplary consensus sequences and enzyme substrates shown in Table 3, can be used to design phosphatase substrate moieties having desired specificities for particular phosphatase and/or phosphatase families, provided that at least one residue is phosphorylated.

The phosphatase substrate moiety can be designed to be reactive with a particular phosphatase or a group of phosphatases, or it can be designed to determine substrate specificity and other catalytic features, such as determining a value for kcat or Km. The phosphorylated residue in the phosphatase substrate moiety can be any group that is capable of being dephosphorylated by a phosphatase. In one embodiment, the residue is a phosphotyrosine residue. In another embodiment, the residue is a phosphoserine residue. In yet another embodiment, the residue is a phosphothreonine residue.

In addition to having one or more phosphorylated residues capable of being dephosphorylated, the phosphatase substrate moiety can include additional amino acid residues (or analogs thereof) that facilitate binding specificity, affinity, and/or rate of dephosphorylation by the phosphatase.

In another aspect, a sulfatase substrate moiety for detecting or characterizing one or more sulfatases in a sample is provided. In some compositions, a substrate molecule comprises a hydrophobic moiety capable of integrating the substrate molecule into a micelle, a substrate moiety comprising a sulphate ester that is capable of being desulfated by a sulfatase, a fluorescent moiety and a charge-balance moiety capable of balancing the overall charge of the micelle, such that the net charge of the micelle ranges from −1 to +1 at physiological pH.

In another exemplary class of compositions, a micelle comprises (i) a substrate molecule that comprises a hydrophobic moiety capable of integrating the substrate molecule into the micelle, a substrate moiety comprising a sulphate ester that is capable of being desulfated by a sulfatase and an optional fluorescent moiety; and (ii) a charge-balance molecule that comprises a hydrophobic moiety capable of integrating the charge-balance molecule into the micelle, a charge-balance moiety capable of balancing the overall charge of the micelle, such that the net charge of the micelle ranges from −1 to +1 at physiological pH. The optional fluorescent moiety, can be part of the substrate molecule, charge-balance molecule, or both.

The sulfatase to be detected can be any sulfatase known in the art. In some embodiments, the sulfatase is a 6-sulfate sulfatase, galactose-6-sulfate sulfatase, galNAc6S sulfatase, chondroitinsulfatase, and chondroitinase. Also, the sulfatase can be a sulfatase candidate, and the method is used to confirm and/or characterize the sulfatase activity of the candidate.

A wide variety of sulfatases have been identified. In some cases, sulfatases are highly specific for only one or a few substrates, but in other cases, sulfatases are relatively non-specific and can act on a large range of substrates including, but not limited to, proteins, glycosaminoglycans, sulfolipids, and steroid sulfates. For example, arylsulphatase A (EC: 3.1.6.8) (ASA), a lysosomal enzyme which hydrolyzes cerebroside sulphate; arylsulphatase B (EC: 3.1.6.12) (ASB), which hydrolyzes the sulphate ester group from N-acetylgalactosamine 4-sulphate residues of dermatan sulphate; arylsulphatase C (ASD) and E (ASE); steryl-sulphatase (EC: 3.1.6.2) (STS), a membrane bound microsomal enzyme which hydrolyzes 3-beta-hydroxy steroid sulphates; iduronate 2-sulphatase precursor (EC: 3.1.6.13) (IDS), a lysosomal enzyme that hydrolyzes the 2-sulphate groups from non-reducing-terminal iduronic acid residues in dermatan sulphate and heparan sulphate; N-acetylgalactosamine-6-sulphatase (EC: 3.1.6.4), which hydrolyzes the 6-sulphate groups of the N-acetyl-d-galactosamine 6-sulphate units of chondroitin sulphate and the D-galactose 6-sulphate units of keratan sulphate; glucosamine-6-sulphatase (EC: 3.1.6.14) (G6S), which hydrolyzes the N-acetyl-D-glucosamine 6-sulphate units of heparan sulphate and keratan sulphate; N-sulphoglucosamine sulphohydrolase (EC: 3.10.1.1) (sulphamidase), the lysosomal enzyme that catalyzes the hydrolysis of N-sulpho-d-glucosamine into glucosamine and sulphate; sea urchin embryo arylsulphatase (EC: 3.1.6.1); green algae arylsulphatase (EC: 3.1.6.1), which plays an important role in the mineralization of sulphates; and arylsulphatase (EC: 3.1.6.1) from Escherichia coli (aslA), Klebsiella aerogenes (gene atsA) and Pseudomonas aeruginosa (gene atsA). In some cases, sulfatases are highly specific for only one target, but in other cases, sulfatases are relatively non-specific and can act on a large range of targets. Accordingly, compositions can be designed to detect particular sulfatases by selection of the sulfatase substrate moiety. Exemplary sulfatases and sulfatase substrates are shown in Table 4, below. These substrates can be used to design sulfatase recognition moieties having desired specificities for particular sulfatases and/or sulfatase families.

TABLE 4

| Sulfatase Description | | |
|---|---|---|
| (Alternative Name(s)) | EC number | Substrate(s) |
| Arylsulfatase | 3.1.6.1 | phenol sulfate |
| (Sulfatase; Aryl-sulphate, | | |
| sulphohydrolase) | | |
| Steryl-sulfatase | 3.1.6.2 | 3-beta-hydroxyandrost-5-en-17-one 3- |
| (Steroid sulfatase; Steryl- | | sulfate and related steryl sulfates |
| sulfate sulfohydrolase; | | |
| Arylsulfatase C) | | |
| Glucosulfatase | 3.1.6.3 | D-glucose 6-sulfate and other sulfates of |
| | | mono- and disaccharides and on adenosine |
| | | 5′-sulfate |
| N-acetylgalactosamine-6- | 3.1.6.4 | 6-sulfate groups of the N-acetyl-D- |
| sulfatase | | galactosamine; 6-sulfate units of chondroitin |
| (Chondroitinsulfatase, | | sulfate and of the D-galactose 6-sulfate units |
| Chondroitinase, Galactose-6- | | of keratan sulfate. |
| sulfate sulfatase) | | |
| Choline-sulfatase | 3.1.6.6 | Choline sulfate |
| Cellulose-polysulfatase | 3.1.6.7 | 2- and 3-sulfate groups of the polysulfates of |
| | | cellulose and charonin |
| Cerebroside-sulfatase | 3.1.6.8 | A cerebroside 3-sulfate; galactose 3-sulfate |
| (Arylsulfatase A) | | residues in a number of lipids; ascorbate 2- |
| | | sulfate; phenol sulfates |
| Chondro-4-sulfatase | 3.1.6.9 | 4-deoxy-beta-D-gluc-4-enuronosyl-(1,4)-N- |
| | | acetyl-D-galactosamine 4-sulfate |
| Chondro-6-sulfatase | 3.1.6.10 | 4-deoxy-beta-D-gluc-4-enuronosyl-(1,4)-N- |
| | | acetyl-D-galactosamine 6-sulfate; N-acetyl- |
| | | D-galactosamine 4,6-disulfate |
| Disulfoglucosamine-6- | 3.1.6.11 | N,6-O-disulfo-D-glucosamine |
| sulfatase | | |
| (N-sulfoglucosamine-6- | | |
| sulfatase) | | |
| N-acetylgalactosamine-4- | 3.1.6.12 | 4-sulfate groups of the N-acetyl-D- |
| sulfatase | | galactosamine; 4-sulfate units of chondroitin |
| (Arylsulfatase B; | | sulfate; dermatan sulfate; N- |
| Chondroitinsulfatase; | | acetylglucosamine 4-sulfate |
| Chondroitinase) | | |
| Iduronate-2-sulfatase | 3.1.6.13 | 2-sulfate groups of the L-iduronate; 2-sulfate |
| (Chondroitinsulfatase) | | units of dermatan sulfate; heparan sulfate |
| | | and heparin. |
| N-acetylglucosamine-6- | 3.1.6.14 | 6-sulfate group of the N-acetyl-D- |
| sulfatase | | glucosamine 6-sulfate; heparan sulfate; |
| (Glucosamine-6-sulfatase; | | keratan sulfate. |
| Chondroitinsulfatase) | | |
| N-sulfoglucosamine-3- | 3.1.6.15 | 3-sulfate groups of the N-sulfo-D- |
| sulfatase | | glucosamine 3-O-sulfate residues of heparin; |
| (Chondroitinsulfatase) | | N-acetyl-D-glucosamine 3-O-sulfate |
| Monomethyl-sulfatase | 3.1.6.16 | Monomethyl sulfate |
| D-lactate-2-sulfatase | 3.1.6.17 | (S)-2-O-sulfolactate |
| Glucuronate-2-sulfatase | 3.1.6.18 | 2-sulfate groups of the 2-O-sulfo-D- |
| (Chondro-2-sulfatase) | | glucuronate residues of chondroitin sulfate, |
| | | heparin and heparitin sulfate. |

The sulfatase substrate moiety can be designed to be reactive with a particular sulfatase or a group of sulfatases, or it can be designed to determine substrate specificity and other catalytic features, such as determining a value for kcat or Km. The sulphate ester in the sulfatase recognition moiety can be any group that is capable of being desulfated by a sulfatase.

In addition to having one or more sulphate esters capable of being desulfated, the sulfatase substrate moiety can include additional groups, for example amino acid residues (or analogs thereof) that facilitate binding specificity, affinity, and/or rate of desulfated by the sulfatase.

In another aspect, a peptidase substrate moiety for detecting, quantifying and/or characterizing one or more protein peptidases in a sample is provided. In some compositions, a substrate molecule comprises a hydrophobic moiety capable of integrating the substrate molecule into a micelle, a substrate moiety comprising a peptide bond that is capable of being hydrolyzed by a peptidase, a fluorescent moiety and a charge-balance moiety capable of balancing the overall charge of the micelle, such that the net charge of the micelle ranges from ⁻1 to ⁺1 physiological pH.

In another exemplary class of compositions, a micelle comprises (i) a substrate molecule that comprises a hydrophobic moiety capable of integrating the substrate molecule into the micelle, a substrate moiety comprising a peptide bond that is capable of being hydrolyzed by a peptidase and an optional fluorescent moiety; and (ii) a charge-balance molecule that comprises a hydrophobic moiety capable of integrating the charge-balance molecule into the micelle, a charge-balance moiety capable of balancing the overall charge of the micelle, such that the net charge of the micelle ranges from −1 to +1 at physiological pH. The optional fluorescent moiety, can be part of the substrate molecule, charge-balance molecule, or both.

A peptidase is any member of a subclass of enzymes of the hydrolase class that catalyze the hydrolysis of peptide bonds. Generally, peptidases are divided into exopeptidases that act only near a terminus of a polypeptide chain and endopeptidases that act internally in polypeptide chains. The peptidase to be detected can be any peptidase known in the art. Also, the peptidase can be a peptidase candidate, and the methods used to confirm and/or characterize the peptidase activity of the candidate.

A wide variety of peptidases have been identified. Generally, peptidases are classified according to their catalytic mechanisms: 1) serine peptidases (such as such as chymotrypsin and trypsin); 2) cysteine peptidases (such as papain); 3) aspartic peptidases (such as pepsin); and, 4) metallo peptidases (such as thermolysin).

In some cases, peptidases are highly specific for only one or a few proteins, but in other cases, peptidases are relatively non-specific and can act on a large range of protein targets. Accordingly, compositions can be designed to detect particular peptidases by suitable selection of the peptidase substrate moiety. Exemplary peptidases and preferential cleavage sites, as indicated by “−/−” are shown in Table 5, below. These various cleavage sites can be used to design peptidase substrate moieties having desired specificities for particular peptidases and/or peptidase families.

TABLE 5

| Peptidase | EC number | Preferential cleavage |
|---|---|---|
| Chymotrypsin. | 3.4.21.1 | Tyr-\|-Xaa, Trp-\|-Xaa, Phe-\|- |
| | | Xaa, Leu-\|-Xaa |
| Trypsin | 3.4.21.4 | Arg-\|-Xaa, Lys-\|-Xaa. |
| Thrombin | 3.4.21.5 | Arg-\|-Gly |
| Renin | 3.4.23.15 | Pro-Phe-His-Leu-\|-Val-Ile |

Xaa - denotes any amino acid

The peptidase substrate moiety can be designed to be reactive with a particular peptidase or a group of peptidases, or it can be designed to determine substrate specificity and other catalytic features, such as determining a value for kcat or Km.

In addition to having one or more peptide bonds capable of being hydrolyzed, the peptidase substrate moiety can include additional amino acid residues (or analogs thereof) that facilitate binding specificity, affinity, and/or rate of hydrolysis by the peptidase.

6.6 The Charge-Balance Moiety

The substrate molecule and/or the charge-balance molecule can further comprise one or more charge-balance moiety(ies). The charge-balance moiety acts to balance the overall charge of the micelle. For example, if the substrate molecule comprises one or more charged chemical groups, the presence of these groups can destabilize the substrate molecule in the micelle, thereby promoting the release of the substrate molecule from the micelle in the absence of the specified enzyme. Release of the charged substrate molecule from the micelle can be prevented by including a charge-balance molecule designed to counter the charge of the substrate molecule via the inclusion of chemical groups that have the opposite charge of the chemical groups comprising the substrate molecule, such that the overall charge of the micelle is approximately neutral. Thus, by including the charge-balance moiety, micelles can be formed in the presence of destabilizing chemical groups.

The charge-balance moiety can be designed to balance the overall charge of the micelle such that net charge of the micelle is about neutral. The overall charge of the micelle depends in part on a number of factors including its chemical composition and pH of the solution comprising the micelle. For example in some embodiments, the substrate molecule comprises a florescent moiety and a substrate moiety, both of which comprise one ore more charged chemical groups that can destabilize or prevent micelle formation. By including a charge-balance molecule that is capable of countering the charge of the substrate molecule, micelles with a net charge between ⁻1 to ⁺1 can be formed at a pH on the range of 6 to 8. Thus, the charge of the charge-balance molecule, depends in part, on the presence of the other charged groups comprising the micelle.

The charge-balance molecule can be designed to have a net negative or net positive charge by including an appropriate number of negatively and positively charged groups in the charge-balance moiety. For example, to establish a net positive charge (i.e., net charge +2), the charge-balance moiety can be designed to contain positively charged groups, or a greater number of positively charged groups than negatively charged groups. To establish a net negative charge (i.e., net charge −2), the charge-balance moiety can be designed to contain negatively charged groups, or a greater number of negatively charged groups than positively charged groups.

The overall charge of the charge-balance molecule also depends in part upon other factors such as the molar ratio of the substrate molecule:charge-balance molecule, the pH of the assay medium, and concentration of salt in the assay medium.

The ratio of charge-balance molecule to substrate molecule can be any ratio capable of balancing the overall charge of the micelle. In some embodiments, the molar ratio between the charge-balance molecule and substrate molecule is 0.5 to 1. In other embodiments, the molar ratio between the charge-balance molecule and substrate molecule is 1 to 1. In other embodiments the molar ratio between the charge-balance molecule and substrate molecule is 1 to 2, or 1 to 5, or 1 to 10. In some embodiments, the molar ratio between the substrate molecule and charge-balance molecule and is 0.5 to 1. In other embodiments, the molar ratio between the substrate molecule and charge-balance molecule is 1 to 1. In other embodiments the molar ratio between the substrate molecule and charge-balance molecule is 1 to 2, or 1 to 5, or 1 to 10.

As another specific example, if the net charge of the substrate molecule is +2, the +2 charge can be balanced by adding an equal molar ratio of a charge-balance molecule with a net charge of −2. In other embodiments, if the net charge of the substrate molecule is +2, the charge can be balanced by adding a charge-balance molecule with a net charge of −1 at a 1:2 molar ratio of substrate molecule to charge-balance molecule.

Another factor effecting the charge of the charge-balance moiety is the pH of the assay medium and the pKas' of the groups comprising the charge-balance moiety. For example, in some embodiments, if the charge-balance moiety is designed to carry a positive charge at pH 7.6, then amino acids with side chains having pKas' above 7.6 can be chosen i.e. lysine (pKa 10.5) and arginine (pKa 12.5) carry a positive charge at pH 7.6. In some embodiments, if the charge-balance moiety is designed to carry a negative charge at pH 7.6, then amino acids with side chains having pKas' below 7.6 can be chosen i.e. aspartic acid (pKa 3.9) and glutamic acid (pKa 4.3) carry a negative charge at pH 7.6. The pKa values of the common amino acids at different pHs are shown in Table 6.

TABLE 6¹

| Amino Acid (IUPAC) | α-COOH pKa | α-NH₃⁺ pKa | Side chain pKa |
|---|---|---|---|
| Alanine (A) | 2.4 | 9.7 | |
| Cysteine (C) | 1.7 | 10.8 | 8.3 |
| Aspartic acid (D) | 2.1 | 9.8 | 3.9 |
| Glutamic acid (E) | 2.2 | 9.7 | 4.3 |
| Phenylalanine (F) | 1.8 | 9.1 | |
| Glycine (G) | 2.3 | 9.6 | |
| Histidine (H) | 1.8 | 9.2 | 6.0 |
| Isoleucine (I) | 2.4 | 9.7 | |
| Lysine (K) | 2.2 | 9.0 | 10.5 |
| Leucine (L) | 2.4 | 9.6 | |
| Methionine (M) | 2.3 | 9.2 | |
| Asparagine (N) | 2.0 | 8.8 | |
| Proline (P) | 2.1 | 10.6 | |
| Glutamine (Q) | 2.2 | 9.1 | |
| Arginine (R) | 2.2 | 9.0 | 12.5 |
| Serine (S) | 2.2 | 9.2 | ˜13 |
| Threonine (T) | 2.6 | 10.4 | ˜13 |
| Valine (V) | 2.3 | 9.6 | |
| Tryptophan (W) | 2.4 | 9.4 | |
| Tyrosine Y | 2.2 | 9.1 | 10.1 |

¹Garerett, R. H. and Grisham M. Biochemistry 2nd edition (1999) Saunders College Publishing. The pKa values depend on temperature, ionic strength, and the microenvironment of the ionizable group.

The charge-balance moiety comprises any group capable of carrying a charge. Suitable examples include amino acids, amino acid analogs, and derivatives, and quartenary compounds such as ammonium and amine compounds.

In some embodiments, the charge-balance moiety can comprise positively charged amino acids such as arginine and lysine. Lysine and arginine contain side chains that carry a single positive charge at physiological pH. The imidazole side chain of histidine has a pKa of about 6, so it carries a full positive charge at a pH of about 6 or less. The charge-balance moiety can comprise negatively charged amino acids such as aspartic acid and glutamic acid. Aspartic acid and glutamic acid contain carboxyl side chains having a single negative charge. Cysteine has a pKa of about 8, so it carries a full negative charge at a pH above 8. The charge-balance moiety can comprise a phosphorylated amino acid. For example, a phosphoserine residue carries two negative charges on a phosphate group.

In some embodiments, the charge-balance moiety can comprise uncharged amino acids such as alanine, asparagine, cysteine, glutamine, glycine, isoleucine, leucine, methionine, phenylalanine, proline, tryptophan, and valine (physiological pH 6 to 8).

In some embodiments, the charge-balance moiety can comprise uncharged amino acids analogs. Suitable examples include 2-amino-4-fluorobenzoic acid, 2-amino-3-methoxybenzoic acid, 3,4-diaminobenzoic acid, 4-aminomethyl-L-phenylalanine, 4-bromo-L-phenylalanine, 4-cyano-L-proline, 3,4,-dihydroxy-L-phenylalanine, ethyl-L-tyrosine, 7-azaatryptophan, 4-aminohippuric acid, 2 amino-3-guanidinopropionic acid, L-citrulline, and derivatives.

In some embodiments, the charge-balance moiety can comprise positively charged amino acids analogs such as N-ω,ω-dimethyl-L-arginine, a-methyl-DL-ornithine, N-ω-nitro-L-arginine, and derivatives.

In some embodiments, the charge-balance moiety can comprise negatively charged amino acids analogs such as 2-aminoadipic acid, N-a-(4-aminobenzoyl)-L-glutamic acid, iminodiacetic acid, a-methyl-L-aspartic acid, a-methyl-DL-glutamic acid, y-methylene-DL-glutamic acid, and derivatives.

In some embodiments, if the substrate moiety comprises an amino acid sequence E-E-I—Y-G-E-F— (SEQ ID NO:32) and a net charge of ⁻3 at pH 7.6, then the charge-balance moiety comprises an amino acid sequence —R—R-E-I—Y-G-R—F— (SEQ ID NO:33) and a net charge of ⁺3 at pH 7.6.

FIG. 1 illustrates an exemplary embodiment of a single molecule embodiment of a substrate molecule comprising hydrophobic moiety R, a fluorescent moiety D, a substrate moiety S and a charge-balance moiety B. The fluorescence of the fluorescent moiety is quenched when the substrate molecule is incorporated into the micelle. The charge-balance moiety act to balance the overall charge of the micelle such that micelle formation is promoted or encouraged. The hydrophobic moiety acts to integrate the substrate molecule(s) of the composition into a micelle when included in an aqueous solvent at or above its critical micelle concentration, thereby quenching the fluorescence fluorescent moiety. The addition of an enzyme that modifies the substrate moiety promotes the dissociation of the fluorescent moiety from the micelle, thereby reducing or eliminating the quenching effect caused by the interactions between the fluorescent moiety and the micelle.

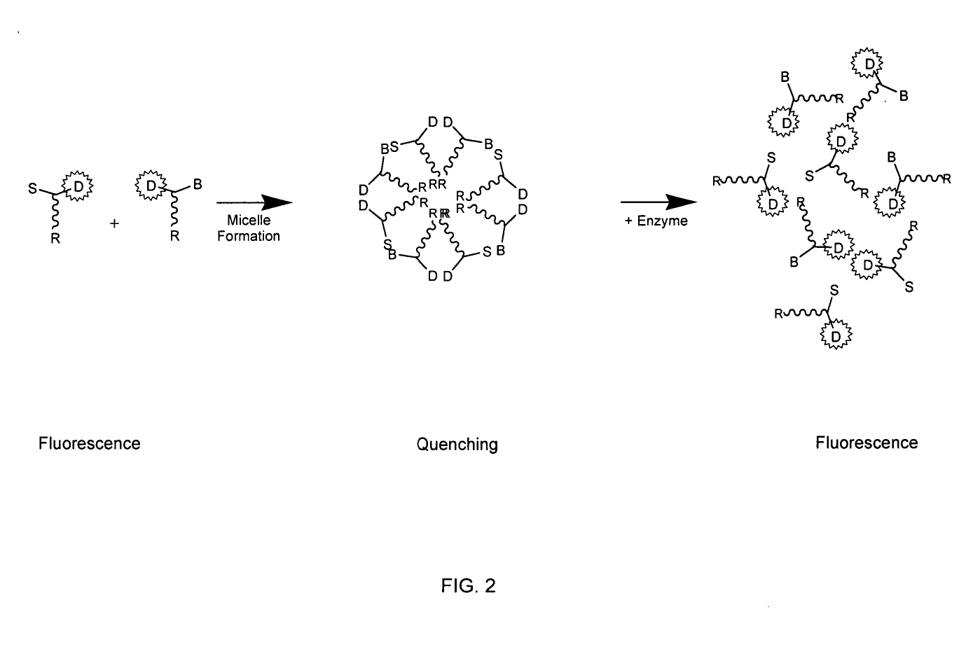

FIG. 2 illustrates an exemplary embodiment wherein the hydrophobic, fluorescent, substrate, and charge-balance moieties are included in two different distinct molecules. The substrate molecule comprises a hydrophobic moiety R, a fluorescent moiety D, and a substrate moiety S. The charge-balance molecule comprises a hydrophobic moiety R, a fluorescent moiety D, and a charge-balance moiety B. The fluorescence of the fluorescent moieties is quenched when the substrate molecule and charge-balance molecule are incorporated into the micelle. The charge-balance moiety act to balance the overall charge of the micelle such that micelle formation is promoted or encouraged. The hydrophobic moieties act to integrate the substrate molecule and the charge-balance molecule of the composition into a micelle when included in an aqueous solvent at or above the critical micelle concentration, thereby quenching the fluorescence of the fluorescent moieties. The addition of an enzyme that modifies the substrate molecule and promotes the dissociation of the fluorescent moieties from the micelle, thereby reducing or eliminating the quenching effect caused by the interactions between the fluorescent moieties and the micelle.

FIG. 3 illustrates an exemplary embodiment wherein the hydrophobic, fluorescent, substrate, charge-balance moieties, and a quenching moiety are included in three different distinct molecules. The quenching molecule comprises a quenching moiety and a hydrophobic moiety. The hydrophobic moiety integrates the quenching molecule into the micelle. The quenching moiety is selected such that it is capable of quenching the fluorescence of a fluorescent moiety of the molecule(s) of the compositions comprising the micelle. If the micelle comprises a plurality of molecules having different fluorescent moieties, a quenching moiety capable of quenching the fluorescence of all or a subset of the fluorescent moieties can be selected. Any of the hydrophobic and quenching moieties previously described can be used to construct a quenching molecule. In other embodiments, the quenching moiety can be part of the substrate molecule or the charge-balance molecule.

In FIG. 3 the substrate molecule comprises a hydrophobic moiety R, a fluorescent moiety D, and a substrate moiety S. The charge-balance molecule comprises a hydrophobic moiety R, a fluorescent moiety D, and a charge-balance moiety B. The quenching molecule comprises a hydrophobic moiety R and a quenching moiety Q. The fluorescence of the fluorescent moieties is quenched when the substrate molecule, charge-balance molecule, and quenching molecule are incorporated into the micelle. The charge-balance moiety act to balance the overall charge of the micelle such that micelle formation is promoted or encouraged. The hydrophobic moieties act to integrate the substrate molecule, the charge-balance molecule, and the quenching molecule of the composition into a micelle when included in an aqueous solvent at or above the critical micelle concentration, thereby quenching the fluorescence of the fluorescent moiety. The addition of an enzyme that modifies the substrate molecule and promotes the dissociation of the fluorescent moieties from the micelle, thereby reducing or eliminating the quenching effect caused by the interactions between the fluorescent moieties and/or quenching moieties and the micelle.

The molar ratio of quenching moiety to fluorescent moiety can be any ratio capable of quenching the fluorescent moiety in the micelle. In some embodiments, the molar ratio between the quenching moiety and fluorescent moiety is 1 to 1. In other embodiments, the molar ratio between the quenching moiety and fluorescent moiety is 1 to 2. In other embodiments the molar ratio between the quenching moiety and fluorescent moiety is 1 to 5, or 1 to 10. In some embodiments, the molar ratio between the fluorescent moiety and quenching moiety is 1 to 2. In other embodiments the molar ratio between the fluorescent moiety and quenching moiety is 1 to 5, or 1 to 10.

The various moieties described herein can be connected in any way that permits them to perform their respective functions. In some embodiments, the various moieties can be connected directly to one another, i.e., covalently linked to each other. In some embodiments, one, some or all of the moieties can be connected indirectly to one another, i.e., via one or more optional linkers.

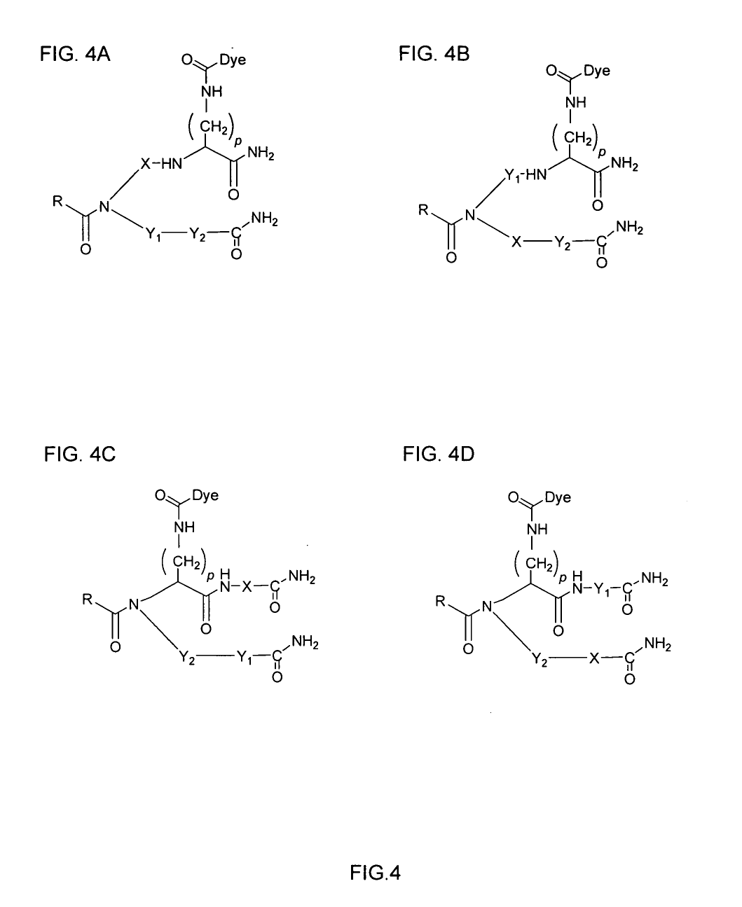

FIGS. 4A-D illustrate exemplary embodiments wherein the hydrophobic, fluorescent, substrate, and charge-balance moieties are included in a single molecule. In the exemplary embodiments depicted in FIGS. 4A-D, hydrophobic moiety R is connected to the remainder of the substrate molecule via a peptide linkage. In some embodiments, the hydrophobic moiety R is linked to the remainder of the substrate molecule via an optional linker. R can comprise any of the hydrophobic moieties described above. In the exemplary embodiments depicted in FIGS. 4A-D, the fluorescent moiety Dye is connected to the remainder of the substrate molecule via a ((CH₂)ₚ—NH—CO—) linkage, wherein p can be any integer form 1 to 6.

FIG. 4A illustrates an exemplary embodiment wherein the charge of the substrate moiety X is balanced by an opposite charge on the charge-balance moiety Y₁. The charge of the fluorescent moiety Dye is balanced by an opposite charge on a second charge-balance moiety Y₂.

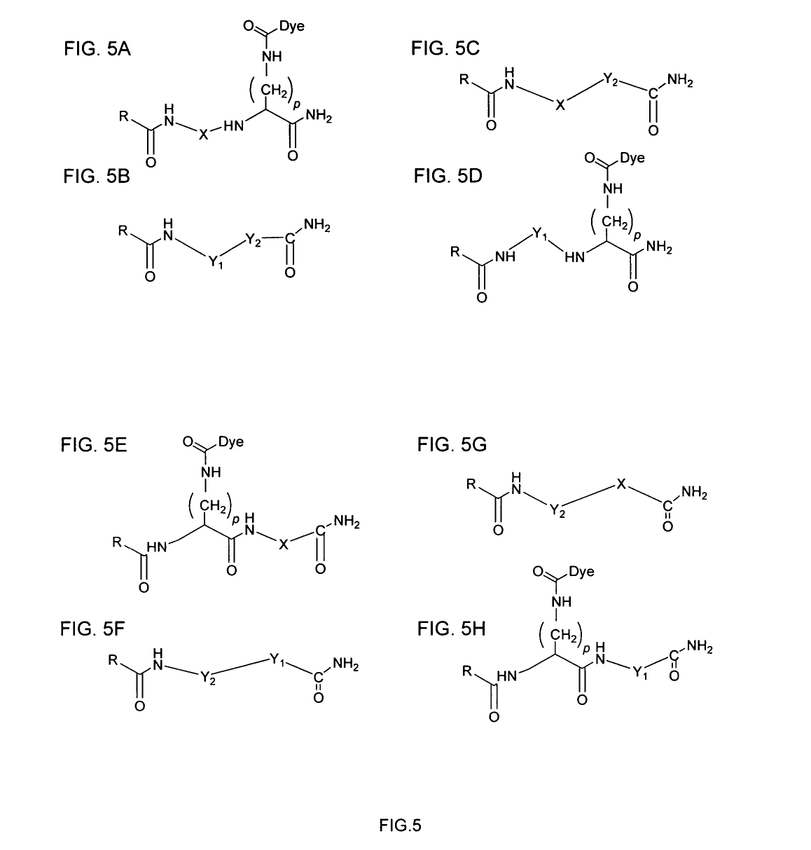

By way of illustration FIGS. 5A-H illustrate exemplary embodiments of compositions comprising two distinct molecules, a substrate molecule (i.e. FIGS. 5A, C, E, G) and a charge-balance molecule (i.e. FIGS. 5B, D, F, H). In the exemplary embodiments depicted in FIGS. 5 A-H, hydrophobic moiety R can comprise any of the hydrophobic moieties described above. In the exemplary embodiments depicted in FIGS. 5A, D, E, and H the substrate molecule and charge-balance molecule comprise the fluorescent moiety Dye.

FIGS. 5A-B illustrate an exemplary embodiment of a composition comprising a substrate molecule and a charge-balance molecule, wherein fluorescent moiety Dye is connected to the substrate moiety X. The charge of the substrate moiety X in the substrate molecule illustrated in FIG. 5A can be balanced by an opposite charge on charge-balance moiety Y₁ in the charge-balance molecule illustrated in FIG. 5B. The charge of the fluorescent moiety Dye in the substrate molecule illustrated in FIG. 5A can be balanced by an opposite charge on charge-balance moiety Y₂ comprising the charge-balance molecule illustrated in FIG. 5B.

FIGS. 5C-D illustrate an exemplary embodiment of a composition comprising a substrate molecule (FIG. 5C) and a charge-balance molecule (FIG. 5D), comprising a fluorescent moiety Dye and charge-balance moiety Y₁. The charge of substrate moiety X in FIG. 5C is balanced by an opposite charge on charge-balance moiety Y₁ in FIG. 5D. The charge of fluorescent moiety Dye in FIG. 5D is balanced by an opposite charge on charge-balance moiety Y₂ in FIG. 5C.

FIGS. 5E-F illustrate an exemplary embodiment of a composition comprising a substrate molecule (FIG. 5E) and a charge-balance molecule (FIG. 5F). The substrate molecule illustrated in FIG. 5E comprises a fluorescent moiety Dye, substrate moiety X and hydrophobic moiety R. The charge of substrate moiety X in FIG. 5E is balanced by an opposite charge on charge-balance moiety Y₁ in FIG. 5F. The charge of fluorescent moiety Dye in FIG. 5E is balanced by an opposite charge on charge-balance moiety Y₂ in FIG. 5F.

FIGS. 5G-H illustrate an exemplary embodiment of a composition comprising a substrate molecule (FIG. 5G) and a charge-balance molecule (FIG. 5H). The substrate molecule illustrated in FIG. 5G comprises a charge balance moiety Y₂, a substrate moiety X, and hydrophobic moiety R. The charge of substrate moiety X in the in FIG. 5G is balanced by an opposite charge on charge-balance moiety Y₁ in FIG. 5H. The charge of fluorescent moiety Dye in FIG. 5H is balanced by an opposite charge on charge-balance moiety Y₂ in FIG. 5G.

In some embodiments optional linkers can be used to link the various moieties of the substrate molecule and the charge-balance molecule. Optional linkers can be provided by one or more (bis)ethylene glycol group(s), also referred to herein as an “O-spacer”. As used herein, each “O-spacer” corresponds to the bracketed illustrated structure. As will be appreciated by a person skilled in the art, the number of oxyethylene units comprising an O-spacer can be selectively varied.

An exemplary example illustrating the use of O-spacers is shown below: 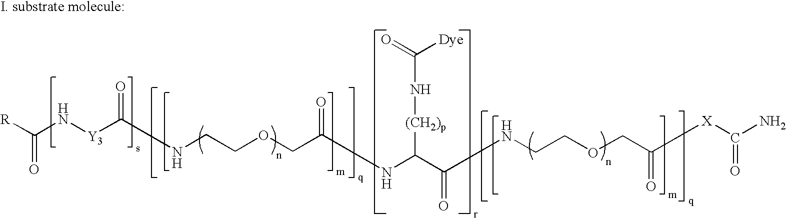 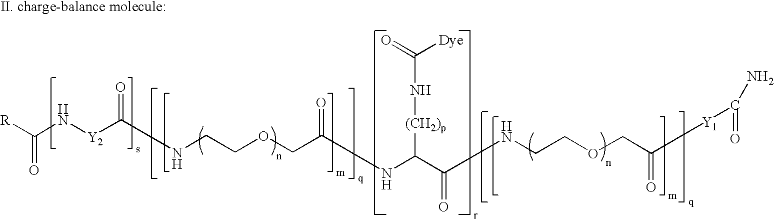
wherein:
R is a hydrophobic moiety;
each s is, independently of the other, 0 or 1;
q represents a linker, each q is, independently of the other, 0 or 1;
m is an integer from 0 to 10; n is an integer from 0 to 10;
r represents a fluorescent moiety, each r is, independently of the other, 0 or 1;
each p is, independently of the other, an integer from 1 to 6;
X comprises a substrate moiety; and
Y₁-Y₃ comprise charge-balance moieties.

Although exemplified with oxyethylene groups, an O-spacer need not be composed of oxyethylene units. Virtually any combination of the same or different oxyethylene units that permits the substrate molecule and charge-balance molecule to function as described herein may be used. In a specific example, an O-spacer may comprise from 1 to about 5 of the same or different lower oxyethylene units (e.g., —(CH₂)ₓCH₂)—, where x is an integer ranging from 0 to 6).

Although O-spacers are illustrated as exemplary optional linkers, the chemical composition of optional linker is not critical for success. The length and chemical composition of the linker can be selectively varied. In some embodiments, the linker can be selected to have specified properties. For example, the linker can be hydrophobic in character, hydrophilic in character, long or short, rigid, semirigid or flexible, depending upon the particular application. The linker can be optionally substituted with one or more substituents or one or more linking groups for the attachment of additional substituents, which may be the same or different, thereby providing a “polyvalent” linking moiety capable of conjugating or linking additional molecules or substances to the signal molecule. In certain embodiments, however, the linker does not comprise such additional substituents or linking groups.

Any molecule having three or more “reactive” groups suitable for attaching other molecule and moieties thereto, or that can be appropriately activated to attach other molecules and moieties thereto could be used to provide a trivalent or higher order multivalent linker. For example, the “backbone” of the multivalent linker to which the reactive linking groups are attached could be linear, branched or cyclic saturated or unsaturated alkyl, a mono or polycyclic aryl or an arylalkyl. Moreover, while the previous examples are hydrocarbons, the multivalent linker backbone need not be limited to carbon and hydrogen atoms. Thus, a multivalent linker backbone can include single, double, triple or aromatic carbon-carbon bonds, carbon-nitrogen bonds, nitrogen-nitrogen bonds, carbon-oxygen bonds, carbon-sulfur bonds and combinations thereof, and therefore can include functionalities such as carbonyls, ethers, thioethers, carboxamides, sulfonamides, ureas, urethanes, hydrazines, etc.

A wide variety of linkers comprised of stable bonds that are suitable for use in the substrates described herein are known in the art, and include by way of example and not limitation, alkyldiyls, substituted alkyldiyls, alkylenos (e.g., alkanos), substituted alkylenos, heteroalkyldiyls, substituted heteroalkyldiyls, heteroalkylenos, substituted heteroalkylenos, acyclic heteroatomic bridges, aryldiyls, substituted aryldiyls, arylaryldiyls, substituted arylaryldiyls, arylalkyldiyls, substituted arylalkyldiyls, heteroaryldiyls, substituted heteroaryldiyls, heteroaryl-heteroaryl diyls, substituted heteroaryl-heteroaryl diyls, heteroarylalkyldiyls, substituted heteroarylalkyldiyls, heteroaryl-heteroalkyldiyls, substituted heteroaryl-heteroalkyldiyls, and the like. Thus, the linker can include single, double, triple or aromatic carbon-carbon bonds, nitrogen-nitrogen bonds, carbon-nitrogen bonds, carbon-oxygen bonds, carbon-sulfur bonds and combinations of such bonds, and may therefore include functionalities such as carbonyls, ethers, thioethers, carboxamides, sulfonamides, ureas, urethanes, hydrazines, etc. In some embodiments, the linker comprises from 1-20 non-hydrogen atoms selected from the group consisting of C, N, O, and S and is composed of any combination of ether, thioether, amine, ester, carboxamide, sulfonamides, hydrazide, aromatic and heteroaromatic groups.

Choosing a linker having properties suitable for a particular application is within the capabilities of those having skill in the art. For example, where a rigid linker is desired, it may comprise a rigid polypeptide such as polyproline, a rigid polyunsaturated alkyldiyl or an aryldiyl, biaryldiyl, arylarydiyl, arylalkyldiyl, heteroaryldiyl, biheteroaryldiyl, heteroarylalkyldiyl, heteroaryl-heteroaryldiyl, etc. Where a flexible linker is desired, it may comprise a flexible polypeptide such as polyglycine or a flexible saturated alkanyldiyl or heteroalkanyldiyl. Hydrophilic linkers may comprise, for example, polyalcohols or polyethers such as polyalkyleneglycols, and O-spacers, as described above. Hydrophobic linkers may comprise, for example, alkyldiyls or aryldiyls.

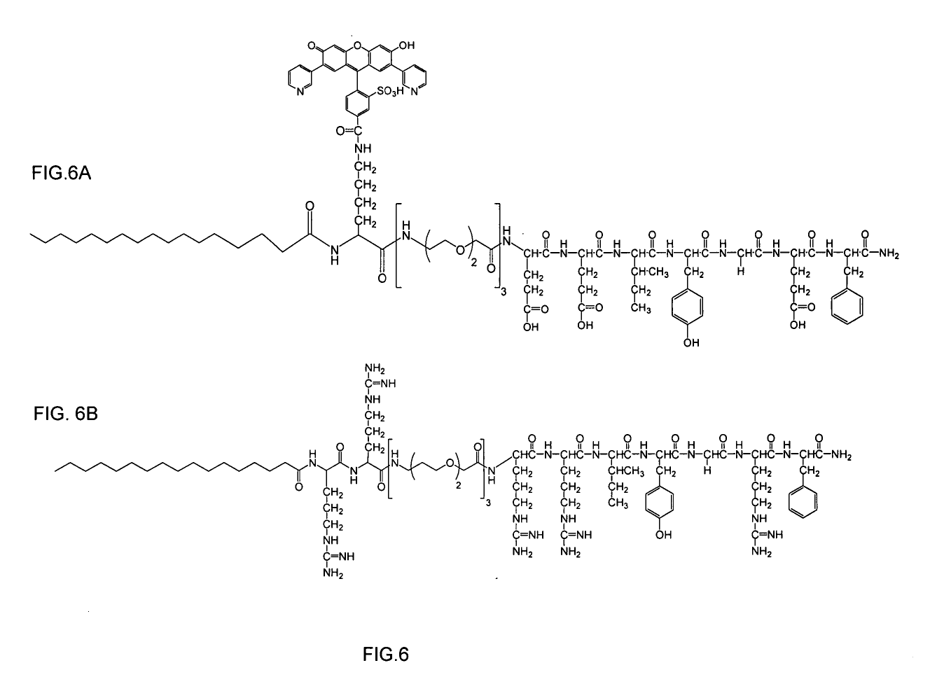

In the exemplary molecules in FIG. 6, the linkage linking the moieties (as well as the linkages linking the optional linker) is a peptide bond. Skilled artisans will appreciate that while using peptide bonds may be convenient, the various moieties comprising the substrates can be linked to one another via any linkage that is stable to the conditions under which the substrates will be used. In some embodiments, the linkages are formed from pairs of complementary reactive groups capable of forming covalent linkages with one another. “Complementary” nucleophilic and electrophilic groups (or precursors thereof that can be suitable activated) useful for effecting linkages stable to biological and other assay conditions are well known. Examples of suitable complementary nucleophilic and electrophilic groups, as well as the resultant linkages formed therefrom, are provided in Table 7

TABLE 7

| Electrophilic Group | Nucleophilic Group | Resultant Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | esters |
| anhydrides | alcohols/phenols | esters |
| anhydrides | amines/anilines | caroboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphate esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | esters |
| sulfonyl halides | amines/anilines | sulfonamides |
| sulfonyl halides | phenols/alcohols | sulfonate esters |

*Activated esters, as understood in the art, generally have the formula —C(O)Z, where Z is, a good leaving group (e.g., oxysuccinimidyl, oxysulfosuccinimidyl, 1-oxybenzotriazolyl, etc.).
**Acyl azides can rearrange to isocyanates.

The hydrophobic, fluorescent, substrate, charge-balance moieties, whether comprising a single molecule or separate molecules, can be connected in any way that permits them to perform their respective functions. In some embodiments, the moieties are connected in a way in to optimize ionic bonding between the charge-balance moiety and the moiety to be balanced. FIG. 6 illustrates exemplary embodiments of a substrate molecule (FIG. 6A) and a charge-balance molecule (FIG. 6B). FIG. 6A illustrates an exemplary substrate molecule that can be used to detect a protein kinase that recognizes a peptide consensus sequence for the tyrosine kinase Lyn, i.e. C₁₆Lys(Dye 2)OOOGluGluIleTyrGlyGluPheNH₂, wherein 000 represents the optional O-spacers, and Dye2 is 5-carboxy-2′,7′-dipyridyl-sulfonefluorescein. In the exemplary embodiment illustrated in FIG. 6A, hydrophobic moiety is a C₁₆ carbon chain and the fluorescent moiety, 5-carboxy-2′,7′-dipyridyl-sulfonefluorescein is linked to the hydrophobic moiety and an optional linker via the amino acid lysine. As will be appreciated by a person of skill in the art, the illustrated lysine is merely an exemplary linker. In FIG. 6A the substrate moiety comprises the peptide sequence Glu-Glu-Ile-Tyr-Gly-Glu-Phe

FIG. 6B illustrates an exemplary charge-balance molecule (i.e. C₁₆ArgArgOOOArgArgIleTyrGlyArgPheNH₂, wherein OOO represents the optional O-spacers) that can be used balance the charge of the substrate molecule illustrated in FIG. 6A. The substrate molecule illustrated in FIG. 6A comprises a fluorescent moiety containing a sulfonate anion with a charge of ⁻2. The substrate molecule illustrated in FIG. 6A further comprises a substrate moiety comprising three glutamate residues, each with a −1 charge. Thus, the total negative charge of the substrate molecule illustrated in FIG. 6A is ⁻5 at physiological pH. The charge-balance molecule illustrated in FIG. 6B comprises guanidinium groups in the five arginine residues, each having a ⁺1 charge. The total positive charge of the charge-balance molecule illustrated in FIG. 6B is ⁺5 at pH 7.6. Thus, the net charge of the compound comprising the substrate molecule illustrated in FIG. 6A and the charge-balance molecule illustrated in FIG. 6B is approximately zero at pH 7.6. Upon phosphorylation of the tyrosine residue by tyrosine kinase Lyn, the net charge of the micelle comprising the substrate molecule and charge-balance molecule is changed from approximately zero to ⁻2, thereby promoting the dissociation of the fluorescent moiety from the micelle, thereby reducing or eliminating the quenching effect and producing a detectable increase in fluorescence.

The various substrate and/or charge-balance molecules can comprise additional moieties. In some embodiments, a substrate molecule can comprise a charge-balance moiety and vice-versa. In some embodiments, the compositions can comprise a quenching moiety.

The substrate molecules and charge-balance molecules can be readily prepared by synthetic methods known in the art. Polypeptides can be prepared by automated synthesizers on a solid support (Perkin J. Am. Chem. Soc. 85:2149-2154 (1963)) by any of the known methods, e.g. Fmoc or BOC (e.g., Atherton, J. Chem. Soc. 538-546 (1981); Fmoc Solid Phase Peptide Synthesis. A Practical Approach, Chan, Weng C. and White, Peter D., eds., Oxford University Press, New York, 2000). Synthetically, polypeptides can be formed by a condensation reaction between the α-carbon carboxyl group of one amino acid and the amino group of another amino acid. Activated amino acids are coupled onto a growing chain of amino acids, with appropriate coupling reagents. Polypeptides can be synthesized with amino acid monomer units where the α-amino group was protected with Fmoc (fluorenylmethoxycarbonyl). Alternatively, the BOC method of peptide synthesis can be practiced to prepare the peptide conjugates of the present teachings.

Amino acids with reactive side-chains can be further protected with appropriate protecting groups. Amino groups on lysine side-chains to be labelled can be protected with an Mtt protecting group, selectively removable with about 5% trifluoroacetic acid in dichloromethane. A large number of different protecting group strategies can be employed to efficiently prepare polypeptides.

Exemplary solid supports include polyethyleneoxy/polystyrene graft copolymer supports (TentaGel, Rapp Polymere GmbH, Tubingen, Germany) and a low-cross link, high-swelling Merrifield-type polystyrene supports with an acid-cleavable linker (Applied Biosystems), although others can be used as well.

Polypeptides are typically synthesized on commercially available synthesizers at scales ranging from 3 to 50 μmoles. The Fmoc group is removed from the terminus of the peptide chain with a solution of piperidine in dimethylformamide (DMF), typically 30% piperidine, requiring several minutes for deprotection to be completed. The amino acid monomer, coupling agent, and activator are delivered into the synthesis chamber or column, with agitation by vortexing or shaking. Typically, the coupling agent is HBTU, and the activator is 1-hydroxybenzotriazole (HOBt). The coupling solution also can contain diisopropylethylamine or another organic base, to adjust the pH to an optimal level for rapid and efficient coupling.

Peptides can alternatively be prepared on chlorotrityl polystyrene resin by typical solid-phase peptide synthesis methods with a Model 433A Peptide Synthesizer (Applied Biosystems, Foster City, Calif.) and Fmoc/HBTU chemistry (Fields, (1990) Int. J. Peptide Protein Res. 35:161-214). The crude protected peptide on resin can be cleaved with 1% trifluoroacetic acid (TFA) in methylene chloride for about 10 minutes. The filtrate is immediately raised to pH 7.6 with an organic amine base, e.g. 4-dimethylaminopyridine. After evaporating the volatile reagents, a crude protected peptide is obtained that can be labelled with additional groups.

Following synthesis, the peptide on the solid support (resin) is deprotected and cleaved from the support. Deprotection and cleavage can be performed in any order, depending on the protecting groups, the linkage between the peptide and the support, and the labelling strategy. After cleavage and deprotection, peptides can be desalted by gel filtration, precipitation, or other means, and analyzed. Typical analytical methods useful for the peptides and peptide conjugates of the present teaching include mass spectroscopy, absorption spectroscopy, HPLC, and Edman degradation sequencing. The peptides and peptide conjugates of the present teachings can be purified by reverse-phase HPLC, gel filtration, electrophoresis, or dialysis.

Fluorescent dyes can be incorporated into the molecules described herein using methods known in the art. For example, a fluorescent dye labeling reagent can bear an electrophilic linking moiety which reacts with a nucleophilic group on the polypeptide, e.g. amino terminus, or side-chain nucleophile of an amino acid. Alternatively, the dye can have a nucleophilic moiety, e.g. amino- or thiol-linking moiety, which reacts with an electrophilic group on the peptide, e.g. NHS of the carboxyl terminus or carboxyl side-chain of an amino acid.

Fluorescent dyes that can be used to prepare the molecules can be prepared synthetically using conventional methods or purchased commercially (e.g. Sigma-Aldrich and/or Molecular Probes). Non-limiting examples of methods that can be used to synthesize suitably reactive fluorescein and/or rhodamine dyes can be found in the various patents and publications discussed above in connection with the fluorescent moiety. Non-limiting examples of suitably reactive fluorescent dyes that are commercially available from Molecular Probes (Eugene, Oreg.) are provided in Table 8, below:

TABLE 8

| Catalog | |
|---|---|
| Number | Product Name |
| C-20050 | 5-carboxyfluorescein-bis-(5-carboxymethoxy-2-nitrobenzyl) |
| | ether,-alanine-carboxamide, succinimidyl ester (CMNB- |
| | caged carboxyfluorescein, SE) |
| C-2210 | 5-carboxyfluorescein, succinimidyl ester (5-FAM, SE) |
| C-1311 | 5-(and-6)-carboxyfluorescein, succinimidyl ester (5(6)-FAM, |
| | SE) |
| D-16 | 5-(4,6-dichlorotriazinyl) aminofluorescein (5-DTAF) |
| F-6106 | 6-(fluorescein-5-carboxamido)hexanoic acid, succinimidyl |
| | ester (5-SFX) |
| F-2182 | 6-(fluorescein-5-(and-6)-carboxamido) hexanoic acid, |
| | succinimidyl ester (5(6)-SFX) |
| F-6129 | 6-(fluorescein-5-(and-6)-carboxamido) hexanoic acid, |
| | succinimidyl ester (5(6)-SFX) |
| F-6130 | fluorescein-5-EX, succinimidyl ester |
| F-143 | fluorescein-5-isothiocyanate (FITC ‘Isomer I’) |
| F-1906 | fluorescein-5-isothiocyanate (FITC ‘Isomer I’) |
| F-1907 | fluorescein-5-isothiocyanate (FITC ‘Isomer I’) |
| F-144 | fluorescein-6-isothiocyanate (FITC ‘Isomer II’) |
| T-353 | Texas Red ® sulfonyl chloride |
| T-1905 | Texas Red ® sulfonyl chloride |
| T-10125 | Texas Red ®-X, STP ester, sodium salt |
| T-6134 | Texas Red ®-X, succinimidyl ester |
| T-20175 | Texas Red ®-X, succinimidyl ester |

6.7 Methods

The compositions find a wide variety of uses in detecting, quantifying and/or characterizing enzymes in biological, medical and industrial applications. The methods generally comprise detecting, quantifying and/or characterizing enzymes in a sample with one or more molecules that collectivity include four different types of moieties: a hydrophobic moiety, a fluorescent moiety, a substrate moiety and a charge-balance moiety.

The sample to be tested can be any suitable sample selected by the user. The sample can be naturally occurring or man-made. For example, the sample can be a blood sample, tissue sample, cell sample, buccal sample, skin sample, urine sample, water sample, or soil sample. The sample can be from a living organism, such as a eukaryote, prokaryote, mammal, human, yeast, or bacterium. The sample can be processed prior to contact with a substrate of the present teachings by any method known in the art. For example, the sample can be subjected to a lysing step, precipitation step, column chromatography step, heat step, etc. In some cases, the sample is a purified or synthetically prepared enzyme that is used to screen for or characterize an enzyme substrate, inhibitor, activator, or modulator.

If the sample contains multiple enzymes, for example both a kinase and a phosphatase, so that the activity of one can interfere with the activity of the other, then an inactivating agent (e.g., an active site directed an irreversible inhibitor) can be added to the sample to inactivate whichever activity is not desired.

The reaction mixture typically includes a buffer, such as a buffer described in the “Biological Buffers” section of the 2000-2001 Sigma Catalog. Exemplary buffers include MES, MOPS, HEPES, Tris (Trizma), bicine, TAPS, CAPS, and the like. The buffer is present in an amount sufficient to generate and maintain a desired pH. The pH of the reaction mixture is selected according to the pH dependency of the activity of the enzyme to be detected, and the charge of the various moieties described herein. For example, the pH can be from 2 to 12, from 5 to 9, or from 6 to 8. The reaction mixture can also contains salts, reducing agents such as dithiothreitol (DTT), and any necessary cofactors and/or cosubstrates for the enzyme (e.g., ATP for a protein kinase, Ca²⁺ ion for a calcium dependent kinase, and cAMP for a protein kinase A). In one embodiment, the reaction mixture does not contain detergent or is substantially free from detergents.

In some embodiments, it can be desirable to dilute the sample to be tested to as low a concentration as reasonably possible to help avoid masking charged groups in the compositions described herein. The sample to be tested can be diluted to any concentration that permits a detectable increase in fluorescence. In some embodiments the sample can be diluted 1, 2, 5, 10, 20, 30, 40, or 50-fold. In some embodiments, a greater 50-fold dilution of the sample can be desirable. In some embodiments the sample can be diluted in the assay reaction mixture.

In some embodiments, it can be desirable to keep the ionic strength as low as reasonably possible to help avoid masking charged groups in the reaction product, so that micelle formation remains disfavored and destabilized. For example, high salt concentration (e.g., 1 M NaCl) can be inappropriate. In addition, it can be desirable to avoid high concentrations of certain other components in the reaction mixture that can also adversely affect the fluorescence properties of the product. Guidance regarding the effects of ionic species, such as metal ions, can be found in Surfactants and Interfacial Phenomena, 2nd Ed., M. J. Rosen, John Wiley & Sons, New York (1989), particularly chapter 3. For example, Mg²⁺ ion at a concentration of 5 mM is useful in the Examples provided below, but higher concentrations can give poorer results.

In practicing certain aspects of the methods, a substrate molecule (or substrate molecule and charge-balance molecule) is mixed with a sample containing an enzyme that is to be detected or that is being used to screen for, detect, quantify, and/or characterize a compound for substrate, inhibitor, activator, or modulator activity. Reaction of the enzyme with the substrate molecule causes an increase (to a more charged species) in the absolute amplitude of the net charge of the micelle, such that the fluorescence of the reacted micelle is greater than the fluorescence of the unreacted micelle. In some embodiments, the a substrate molecule (or substrate molecule and charge-balance molecule) has a net charge of zero (neutral net charge), and reaction of the substrate molecule with the enzyme makes the substrate molecule either (1) net negatively charged by (1A) adding or generating a new negatively charged group on the substrate moiety, or (1B) removing or blocking a positively charged group on the substrate moiety; or (2) net positively charged, by (2A) adding or generating a new positively charged group on the substrate moiety, or (2B) removing or blocking a negatively charged group on the substrate moiety.

For example, reaction (1A) can be accomplished by adding a phosphate group to a hydroxyl group on the substrate moiety (changing a neutrally charged group to a group having a charge of −2, (e.g., using a protein kinase), by cleaving a carboxylic ester or amide to produce a carboxyl group (changing a neutrally charged group to a group having a charge of −1, e.g., using an esterase or amidase). Reaction (1B) can be accomplished by cleaving a positively charge amino acids, or can be accomplished by reacting an amino or hydrazine group in the enzyme recognition moiety with an acetylating enzyme to produce a neutral acetyl ester group, with an N-oxidase enzyme to produce a neutral N-oxide, with an ammonia lyase to remove ammonia, or with an oxidase that causes oxidative deamination, for example. Reaction (2A) can be accomplished, for example, by treating an amide group in the substrate moiety with an amidase to generate a positively charged amino group in the substrate molecule. Reaction (2B) can be accomplished by cleaving a negativity charge amino acids, or can be accomplished using a decarboxylase enzyme to remove a carboxylic acid or by reacting a carboxyl group with a methyl transferase to form a carboxylic ester, for example. A variety of enzymes capable of performing such transformations are known in the literature (e.g., see C. Walsh, Enzymatic Reaction Mechanisms, WH Freeman and Co., New York, (1979), the Worthington Product Catalog (Worthington Enzymes), Sigma Life Sciences Catalog, and the product catalogs of other commercial enzyme suppliers).

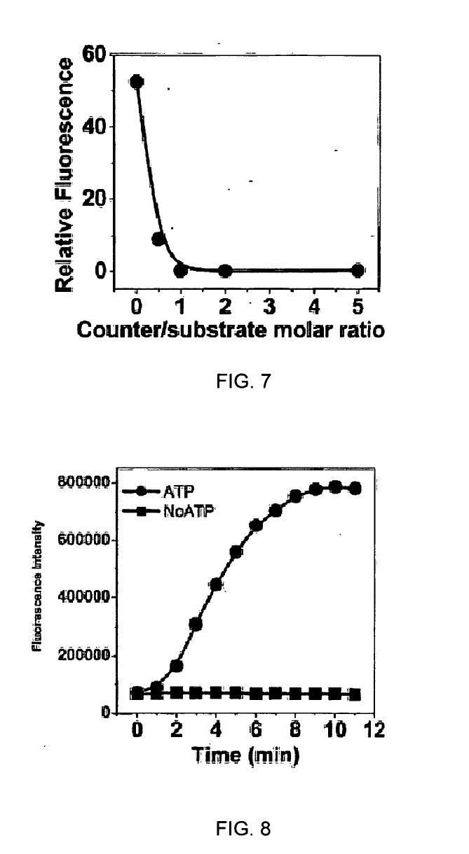

While the basis for increased fluorescence is not certain, and the inventors do not wish to be bound to a particular theory, it is contemplated that the fluorescent substrate molecule and/or charge-balance molecule of the present teachings are capable of forming micelles in the reaction mixture due to the hydrophobic moiety(ies), so that the fluorescent moieties quench each other due to their close proximity. Micelle formation can be particularly favored when the charge on the substrate molecule is balanced by the charge on the charge-balance moiety(ies) so that the net charge is approximately zero, or a small negative or small positive net charge, so that micelle formation is not prevented by mutual charge repulsion. While not intending to be bound by any theory of operation, it is believed that ionic bonds can be formed between oppositely charged charge-balance moiety(ies) and any other moieties described herein in aqueous solution at physiological pH and promote or encourage micelle formation. For example, FIG. 7 shows that the addition of varying concentrations (0, 5, 10, 20, 50 μM) of a charge-balance molecule, C₁₆RROOORRIYGRF quenches the fluorescence of a substrate molecule, C₁₆K(Dye2)OOOEEIYGEF (10 μM) in 25 mM Tris (pH 7.6). While not intending to be bound by any theory of operation, it is contemplated that the fluorescent substrate molecule and charge-balance molecule are capable of forming micelles so that the fluorescent moieties quench each other due to their close proximity.

To be effective, not only should a compound comprising a substrate molecule and charge-balance molecule react with the enzyme to form the desired modified product, but also the product should be more fluorescent than the compound comprising the substrate molecule and charge-balance molecule, so that a detectable increase in fluorescence can be observed. Generally, a greater change in fluorescence provides greater assay sensitivity, provided that an adequately low signal-to-noise ratio is achieved. Therefore, it can be desirable to test multiple molecules comprising a hydrophobic moiety, a fluorescent moiety, a substrate moiety and a charge-balance moiety to find a molecule having the most suitable fluorescence properties.

The rate of the reaction for a tyrosine kinase using 2 μM substrate molecule (C₁₆Lys(Dye 2)OOOGluGluIleTyrGlyGluPheNH₂) and 2 μM charge-balance molecule (C₁₆ArgArgOOOArgArgIleTyrGlyArgPheNH₂), and 0 or 100 μM ATP, and 5 nM tyrosine kinase Lyn is shown in FIG. 8. The addition of tyrosine kinase Lyn to the micelle comprising the substrate molecule and charge-balance molecule cause an increase in fluorescence over time.

The present disclosure contemplates not only detecting enzymes, but also methods involving: (1) screening for and/or quantifying enzyme activity in a sample, (2) determining kcat and/or Km of an enzyme or enzyme mixture with respect to selected substrates, (3) detecting, screening for, and/or characterizing substrates of enzymes, (4) detecting, screening for, and/or characterizing inhibitors, activators, and/or modulators of enzyme activity, and (5) determining substrate specificities and/or substrate consensus sequences or substrate consensus structures for selected enzymes.

For example, in screening for enzyme activity, a sample that contains, or can contain, a particular enzyme activity is mixed with a substrate of the present teachings, and the fluorescence is measured to determine whether an increase in fluorescence has occurred. Screening can be performed on numerous samples simultaneously in a multi-well or multi-reaction plate or device to increase the rate of throughput. Kcat and Km can be determined by standard methods, as described, for example, in Fersht, Enzyme Structure and Mechanism, 2nd Edition, W.H. Freeman and Co., New York, (1985)).

In some embodiments, the reaction mixture can contain two or more different enzymes. This can be useful, for example, to screen multiple enzymes simultaneously to determine if an enzyme has a particular enzyme activity.

The substrate specificity of an enzyme can be determined by reacting an enzyme with different substrate molecules having different substrate moieties the activity of the enzyme toward the substrates can be determined based on an increase in fluorescence. For example, by reacting an enzyme with several different substrate molecules having several different protein kinase recognition moieties, a consensus sequence for preferred substrates of a kinase can be prepared.

Although not necessary for operation of the methods, the assay mixture can optionally include one or more quenching moieties or quenching molecules designed to quench the fluorescence of the fluorescent moiety of the substrate molecule and/or charge-balance molecule.

Detecting, screening for, and/or characterizing inhibitors, activators, and/or modulators of enzyme activity can be performed by forming reaction mixtures containing such known or potential inhibitors, activators, and/or modulators and determining the extent of increase or decrease (if any) in fluorescence signal relative to the signal that is observed without the inhibitor, activator, or modulator. Different amounts of these substances can be tested to determine parameters such as Ki (inhibition constant), K_{H} (Hill coefficient), Kd (dissociation constant) and the like to characterize the concentration dependence of the effect that such substances have on enzyme activity.

Detection of fluorescent signal can be performed in any appropriate way. Advantageously, substrate molecules/charge-balance molecules of the present teachings can be used in a continuous monitoring phase, in real time, to allow the user to rapidly determine whether enzyme activity is present in the sample, and optionally, the amount or specific activity of the enzyme. The fluorescent signal is measured from at least two different time points, usually until an initial velocity (rate) can be determined. The signal can be monitored continuously or at several selected time points. Alternatively, the fluorescent signal can be measured in an end-point embodiment in which a signal is measured after a certain amount of time, and the signal is compared against a control signal (before start of the reaction), threshold signal, or standard curve.

6.8 Kits

Also provided are kits for performing methods of the present teachings. The kits generally comprise one or more molecules that collectivity include four different types of moieties: a hydrophobic moiety, a fluorescent moiety, a substrate moiety and a charge-balance moiety.

In one embodiment, the kit comprises a substrate molecule for detecting a target enzyme, and a buffer for preparing a reaction mixture that facilitates the enzyme reaction. In another embodiment, the kit comprises a substrate molecule for detecting a target enzyme, a charge-balance molecule, and a buffer for preparing a reaction mixture that facilitates the enzyme reaction. The buffer can be provided in a container in dry form or liquid form. The choice of a particular buffer can depend on various factors, such as the pH optimum for the enzyme to be detected, the solubility and fluorescence properties of the fluorescent moiety in the substrate molecule and/or charge-balance molecule, and the pH of the sample from which the target enzyme is obtained. The buffer is usually added to the reaction mixture in an amount sufficient to produce a particular pH in the mixture. In some embodiments, the buffer is provided as a stock solution having a pre-selected pH and buffer concentration. Upon mixture with the sample, the buffer produces a final pH that is suitable for the enzyme assay, as discussed above. The pH of the reaction mixture can also be titrated with acid or base to reach a final, desired pH. The kit can additionally include other components that are beneficial to enzyme activity, such as salts (e.g., KCl, NaCl, or NaOAc), metal salts (e.g., Ca2+ salts such as CaCl₂, MgCl₂, MnCl₂, ZnCl₂, or Zn(OAc), detergents (e.g., TWEEN 20), and/or other components that can be useful for a particular enzyme. These other components can be provided separately from each other or mixed together in dry or liquid form.

The molecules that collectivity include four different types of moieties: a hydrophobic moiety, a fluorescent moiety, a substrate moiety and a charge-balance moiety can be provided in dry or liquid form, together with or separate from the buffer. To facilitate dissolution in the reaction mixture, the substrate molecule and/or charge-balance molecule can be provided in an aqueous solution, partially aqueous solution, or non-aqueous stock solution that is miscible with the other components of the reaction mixture. For example, in addition to water, a substrate solution can also contain a cosolvent such as dimethyl formamide, dimethylsulfonate, methanol or ethanol, typically in a range of 1%-10% (v:v).

The kit can also contain additional chemicals useful in the detection, quantifying, and/or characterizing of enzymes. For example, for the detection of protein kinase activity, the kit can also contain a phosphate-donating group, such as ATP, GTP, ITP (inosine triphosphate) or other nucleotide triphosphate or nucleotide triphosphate analog that can be used by the kinase to phosphorylate the substrate moiety.

The operation of the various compositions and methods can be further understood in light of the following non-limiting examples that illustrate various aspects of the present teachings.

### 7. EXAMPLES

Aspects of the present teachings may be further understood in light of the following examples, which should not be construed as limiting the scope of the present teachings in any way.

7.1 Preparation of Substrate Molecules and Charge-Balance Molecules

Resins and reagents for peptide synthesis, Fmoc amino acids, 5-carboxyfluorescein succinimidyl ester were obtained from Applied Biosystems (Foster City, Calif.). Fmoc-Lys(Mtt)-OH, Fmoc-Ser(OPO(OBzl(OH)—OH and Fmoc-Dpr(ivDde) were obtained from Novabiochem. All other chemicals and buffers were obtained from Sigma/Aldrich.

Peptide synthesis was performed on an Applied Biosystems Model 433A Peptide Synthesizer. HPLC was performed on an Agilent 1100 series HPLC. UV-Vis measurements were performed on a Cary 3E UV-Vis spectrophotometer. MALDI Mass spectral data were obtained on an Applied Biosystems Voyager using cyano-4-hydroxycinnamic acid as matrix material.

An exemplary substrate molecule useful for detecting protein tyrosine kinase Lyn, C₁₆Lys(Dye 2)OOOGluGluIleTyrGlyGluPheNH₂ was prepared as follows. The peptide OOOK(ivDde)GluGluIleTyrGlyGluPhe(Mtt) was constructed via solid phase peptide synthesis using standard FastMoc™ chemistry on 125 mg of Fmoc-PAL-PEG-PS resin at 0.16 mmol/g, a solid support which results in a carboxamide peptide. A portion of the final protected peptide-resin (20 mg, 2 μmol peptide) was transferred to a 1.5 ml eppendorf tube and treated with 1 mL of 5% trifluoroacetic acid (TFA) in dichloromethane (DCM), giving a characteristic yellow trityl color. The resin was treated with additional 1 mL portions of 5% TFA until the washes were colorless. The resin was washed with DCM (1 mL). Dodecanoic acid (20 mg), HBTU/HOBT solution (0.1 mL) and diisopropylethylamine (0.04 mL) were added to the resin and the mixture was agitated gently for 20 min. The resin was washed with DMF (5×1 mL) and treated with 10% hydrazine in DMF for ten minutes. 5-Carboxy-2′,7′-dipyridylsulfonefluorescein (10 mg), HBTU/HOBT solution (0.1 mL) and diisopropylethylamine (0.04 mL) were added to the resin and the mixture agitated for 45 minutes. The resin was washed with 8×1 mL DMF, 1×1 mL acetonitrile. The peptide was cleaved from the resin with 1 mL cleavage solution (950 μL TFA, 50 μL water). After 1.5 to 2 h the mixture was filtered and the filtrate concentrated to dryness on a rotary evaporator. The residue was dissolved in water (0.5 mL) and a portion purified by reverse-phase HPLC (Metachem Technologies column: 150×4.6 mm, Polaris C18, 5 um) using a 30% to 70% gradient over 10 min of 0.1% TFA in acetonitrile vs. 0.1% TFA in water. The dye-labeled peptide was analyzed by MALDI mass spectrometry, which resulted in the expected M/z=2234. The peptide solution was evaporated to dryness, redissolved in water, and quantitated. The extinction coefficient of 5-Carboxy-2′,7′-dipyridylsulfonefluorescein was assumed to be 80,000 cm⁻¹M⁻¹.B

An exemplary charge-balance molecule C₁₆ArgArgOOOArgArgIleTyrGlyArg PheNH₂ useful for balancing the charge of substrate molecule C₁₆Lys(Dye 2)OOOGluGluIleTyrGlyGluPheNH₂, was prepared as follows. The peptide ArgArgOOOArgArgIleTyrGlyArgPheNH₂ (Mtt) was constructed via solid phase peptide synthesis using standard FastMoc™ chemistry on 125 mg of Fmoc-PAL-PEG-PS resin at 0.16 mmol/g, a solid support which results in a carboxamide peptide. A portion of the final protected peptide-resin (20 mg, 2 μmol peptide) was transferred to a 1.5 ml eppendorf tube and treated with 1 mL of 5% trifluoroacetic acid (TFA) in dichloromethane (DCM), giving a characteristic yellow trityl color. The resin was treated with additional 1 mL portions of 5% TFA until the washes were colorless. The resin was washed with DCM (1 mL). Dodecanoic acid (20 mg), HBTU/HOBT solution (0.1 mL) and diisopropylethylamine (0.04 mL) were added to the resin and the mixture was agitated gently for 20 min. The resin was washed with DMF (5×1 mL) and treated with 10% hydrazine in DMF for ten minutes. 5-Carboxy-2′,7′-dipyridylsulfonefluorescein (10 mg), HBTU/HOBT solution (0.1 mL) and diisopropylethylamine (0.04 mL) were added to the resin and the mixture agitated for 45 minutes. The resin was washed with 8×1 mL DMF, 1×1 mL acetonitrile. The peptide was cleaved from the resin with 1 mL cleavage solution (950 μL TFA, 50 μL water). After 1.5 to 2 h the mixture was filtered and the filtrate concentrated to dryness on a rotary evaporator. The residue was dissolved in water (0.5 mL) and a portion purified by reverse-phase HPLC (Metachem Technologies column: 150×4.6 mm, Polaris C18, 5 um) using a 30% to 70% gradient over 10 min of 0.1% TFA in acetonitrile vs. 0.1% TFA in water. The peptide was analyzed by MALDI mass spectrometry, which resulted in the expected M/z=1952. The peptide solution was evaporated to dryness, redissolved in water, and quantitated.

7.2 Addition of Charge-Balance Molecule Quenches the Fluorescence of the Substrate Molecule

A reaction solution was prepared containing 10 M substrate molecule C16Lys(Dye 2)OOOGluGluIleTyrGlyGluPheNH2 and 25 mM Tris (pH 7.6), 5 mM MgCl and 5 mM DTT. Varying concentrations of the charge-balance molecule C16ArgArgOOOArgArgIleTyrGlyArg PheNH2 were added (final concentration 0, 5 μM, 10 μM, 20 μM, 50 μM) and the fluorescence was determined. The results are shown in FIG. 7.

7.3 Detection of Protein Kinase Activity

Kinase assays were performed using Corning 384-well, black, non-binding surface (NBS), microwell plates. Fluorescence was read in real time using a Molecular Dynamics Gemini XS plate reader, with excitation and emission set at 500 and 550 respectively. The plate was read every minute for one hour at ambient temperature.

Concentrations of the substrate molecule C16Lys(Dye 2)OOOGluGluIleTyrGlyGluPheNH2 and charge-balance molecule C16ArgArgOOOArgArgIleTyrGlyArg PheNH2 were determined by dilution of the purified peptides into dimethylformamide (200 □L) with 1 M NaOH (5 □L) and measuring the absorbance of 5-carboxy-2′,7′-dipyridyl-sulfonefluorescein (Dye2) at its absorbance maximum (548 nm). The extinction coefficient of Dye2 was assumed to be 80,000 cm1M-1.

A reaction solution was prepared containing the substrate molecule C₁₆Lys(Dye 2)OOOGluGluIleTyrGlyGluPheNH₂ (2 μM), and charge-balance molecule C₁₆ArgArgOOOArgArgIleTyrGlyArg PheNH₂ (2 μM), 20 mM Tris buffer, pH 7.6, MgCl₂ (5 mM), DTT (5 mM) and Lyn (5 nM). The solution was pipetted into wells of a 384-well plate (10 mL per well). ATP (0 or 100 μM) was added to initiate the kinase reaction. The plate was read at 500 nm excitation, 550 nm emission, each minute for 1 hour. The results are shown in FIG. 8.

All publications and patent applications mentioned herein are hereby incorporated by reference as if each publication or patent application was specifically and individually indicated to be incorporated by reference.

The section headings used herein are for organizational purposes only and are not to be construed as limiting the subject matter described in any way.

While the present teachings are described in conjunction with various embodiments, it is not intended that the present teachings be limited to such embodiments. On the contrary, the present teachings encompass various alternatives, modifications, and equivalents, as will be appreciated by those skilled in the art.

1. A substrate molecule comprising a hydrophobic moiety capable of integrating the substrate molecule into a micelle, a substrate moiety, a fluorescent moiety and a charge-balance moiety capable of balancing the overall charge of the micelle, such that the net charge of the micelle ranges from −1 to +1 at physiological pH.

2. The substrate molecule of claim 1, which has a net charge of zero at physiological pH.

3. The substrate molecule of claim 1 in which the hydrophobic moiety comprises a hydrocarbon containing from 6 to 30 carbon atoms.

4. The substrate molecule of claim 3 in which the hydrocarbon is a saturated or unsaturated alkyl.

5. The substrate molecule of claim 1 in which the fluorescent moiety is capable of self-quenching.

6. The substrate molecule of claim 1 in which the fluorescent moiety comprises a xanthene dye.

7. The substrate molecule of claim 5 in which the xanthene dye is selected from a fluorescein dye and a rhodamine dye.

8. The substrate molecule of claim 1 that further comprises a quenching moiety capable of quenching the fluorescence of the fluorescent moiety.

9. The substrate molecule of claim 1 in which the substrate moiety comprises a substrate acted upon by a kinase.

10. The substrate molecule of claim 1 in which the substrate moiety comprises a peptide sequence made entirely or in part of:

| -R-R-X-S/T-Z- | (SEQ ID NO: |
|---|---|
| | 1) |
| -L-R-R-A-S-L-G- | (SEQ ID NO: |
| | 2) |
| -R-X-X-S/T-F-F- | (SEQ ID NO: |
| | 3) |
| -R-Q-G-S-F-R-A- | (SEQ ID NO: |
| | 4) |
| -S/T-P-X-R/K- | (SEQ ID NO: |
| | 5) |
| -P-X-S/T-P- | (SEQ ID NO: |
| | 6) |
| -R-R-I-P-L-S-P- | (SEQ ID NO: |
| | 7) |
| -K-K-K-K-R-F-S-F-K- | (SEQ ID NO: |
| | 8) |
| -X-R-X-X-S-X-R-X- | (SEQ ID NO: |
| | 9) |
| -L-R-R-L-S-D-S-N-F- | (SEQ ID NO: |
| | 10) |
| -K-K-L-N-R-T-L-T-V-A- | (SEQ ID NO: |
| | 11) |
| -E-E-I-Y-E/G-X-F- | (SEQ ID NO: |
| | 12) |
| -E-E-I-Y-G-E-F-R- | (SEQ ID NO: |
| | 13) |
| -E-I-Y-E-X-I/V- | (SEQ ID NO: |
| | 14) |
| -I-Y-M-F-F-F- | (SEQ ID NO: |
| | 15) |
| -Y-M-M-M- | (SEQ ID NO: |
| | 16) |
| -E-E-E-Y-F- | (SEQ ID NO: |
| | 17) |
| -R-I-G-E-G-T-Y-G-V-V-R-R- | (SEQ ID NO: |
| | 18) |
| -R-P-R-T-S-S-F- | (SEQ ID NO: |
| | 19) |
| -P-R-T-P-G-G-R- | (SEQ ID NO: |
| | 20) |
| -R-L-N-R-T-L-S-V- | (SEQ ID NO: |
| | 21) |
| -D-R-R-L-S-S-L-R- | (SEQ ID NO: |
| | 22) |
| -E-A-I-Y-A-A-P-F-A-R-R-R- | (SEQ ID NO: |
| | 23) |
| -K-V-E-K-I-G-E-G-T-Y-G-V-V-Y-K | (SEQ ID NO: |
| | 24) |
| -E-E-E-I-Y-G-E-F- | (SEQ ID NO: |
| | 25) |
| -R-H-S-S-P-H-Q-S(PO42-)-E-D-E-E- | (SEQ ID NO: |
| | 26) |
| -R-R-K-D-L-H-D-D-E-E-D-E-A-M-S-I-T-A | (SEQ ID NO: |
| | 27) |
| -S(PO42-)-X-X-S/T- | (SEQ ID NO: |
| | 28) |
| -S-X-X-E/D- | (SEQ ID NO: |
| | 29) |
| -R-R-R-D-D-D-S-D-D-D- | (SEQ ID NO: |
| | 30) |
| -K-G-P-W-L-E-E-E-E-E-A-Y-G-W-L-D-F- | (SEQ ID NO: |
| | 31); |

or any combination thereof; and analogs and conservative mutants thereof, wherein X represents any residue, Z represents a hydrophobic residue, and S(PO42—) represents a phosphorylated residue.

11. The substrate molecule of claim 1 in which the substrate moiety comprises a substrate acted upon by a phosphatase, sulfatase, or peptidase.

12. The substrate molecule of claim 1 in which the charge-balance moiety comprises amino acids having charged side chain groups.

13. The substrate molecule of claim 1 in which the substrate moiety comprises the peptide sequence -E-E-I—Y-G-E-F— (SEQ ID NO:32) and the charge-balance moiety comprises the peptide sequence —R—R-E-I—Y-G-R—F— (SEQ ID NO:33).

14. A charge-balance molecule comprising a hydrophobic moiety capable of integrating the charge-balance molecule into a micelle, a fluorescent moiety, and a charge-balance moiety having a charge at physiological pH.

15. A micelle comprising a hydrophobic moiety, a fluorescent moiety, a substrate moiety and a charge-balance moiety capable of balancing the overall charge of the micelle, such that the net charge of the micelle ranges from −1 to +1 at physiological pH, wherein the fluorescence of the fluorescent moiety is quenched.

16. The micelle of claim 15 in which the hydrophobic moiety, fluorescent moiety, substrate moiety and charge-balance moieties are contained within a single molecule.

17. The micelle of claim 15 comprising: (i) a substrate molecule that comprises a hydrophobic moiety capable of integrating the substrate molecule into the micelle, a substrate moiety and an optional fluorescent moiety; and (ii) a charge-balance molecule that comprises a hydrophobic moiety capable of integrating the charge-balance molecule into the micelle, a charge-balance moiety capable of balancing the overall charge of the micelle, such that the net charge of the micelle ranges from −1 to +1 at physiological pH, and an optional fluorescent moiety, wherein one or both of the substrate and charge-balance molecules includes the optional fluorescent moiety.

18-29. (canceled)

30. A method of detecting and/or characterizing an enzyme activity in a sample, comprising the steps of: (i) contacting the sample with a substrate molecule according to claim 1, under conditions effective to permit the enzyme, when present in the sample, to modify the substrate molecule in a manner that leads to an increase in a fluorescence signal produced by a fluorescent moiety; and (ii) detecting a fluorescence signal, where an increase in the fluorescence signal indicates the presence and/or quantity of the enzyme in the sample.

31. (canceled)

32. A kit for detecting and/or characterizing an enzyme activity in a sample comprising: a substrate molecule comprising a hydrophobic moiety capable of integrating the substrate molecule into a micelle, a substrate moiety, a fluorescent moiety and a charge-balance moiety capable of balancing the overall charge of the micelle, such that the net charge of the micelle ranges from −1 to +1 at physiological pH.

33. (canceled)
